(19) United States
(12) Patent Application Publication
(10) Pub. No.: US 2018/0328848 A1
(43) Pub. Date: Nov. 15, 2018

(54) CELL DETECTION, CAPTURE, ANALYSIS, AGGREGATION, AND OUTPUT METHODS AND APPARATUS

(71) Applicant: AXON DX, LLC, Earlysville, VA (US)

(72) Inventors: Kent A. MURPHY, Arlington, VA (US); Philip R. COUCH, Honiton (GB); Jeffrey A. SMITH, Earlysville, VA (US)

(21) Appl. No.: 15/772,889

(22) PCT Filed: Nov. 2, 2016
(86) PCT No.: PCT/US2016/060010

§ 371 Date: May 2, 2018

Related U.S. Application Data
(60) Provisional application No. 62/250,534, filed on Nov. 4, 2015.
(60) Provisional application No. 62/365,489, filed on Jul. 22, 2016.

(51) Int. Cl. G01N 21/64; G02B 21/16; G02B 21/36; G06K 9/00

(52) U.S. Cl. CPC G01N 21/6458; G02B 21/16; G02B 21/365; G01N 33/5091; G01N 2021/6419; G01N 2021/6441; G01N 2201/0627; G06K 9/00134

(57) ABSTRACT

An optical system is provided for clinical diagnostics that include methods and apparatus for rapidly detecting and characterizing rare cell objects in a biological sample. The sample is processed, loaded onto a “capture zone” in the optical system, and subjected to a two-stage optical process for very rapid detection and detailed characterization of detected cells and cell fragments. Detected rare cells and rare cell fragments are characterized with regards to biomarker profiles using fluorescent tags or chromophores. A sample is scanned in a first time period to generate a first set of image data from which marked cell objects are detected. The marked cell objects are ranked, and respective area or volume values are determined for the ranked cell objects. The respective area or volume values for the ranked cell objects are combined to generate an equivalent cell count. In some embodiments, a total cell count for the sample is determined based on the equivalent cell count, and in other embodiments, it is determined based on the equivalent cell count and a count of detected marked cell objects. In other embodiments, aggregate information for detected marked cell objects for the sample is determined and output as an indicator of progression of disease.

### PRIORITY APPLICATIONS

Priority is claimed to U.S. provisional patent application 62/365,489, filed on Jul. 22, 2016, and to U.S. provisional patent application 62/250,534, filed on Nov. 4, 2015, the contents of both of which are incorporated herein by reference.

### INTRODUCTION

Circulating tumor cells in the blood stream play a critical role in establishing metastases. The clinical value of CTCs as a biomarker for early cancer detection, diagnosis, prognosis, prediction, stratification, and pharmacodynamics have been widely explored in recent years. However, the clinical utility of current CTC tests is limited mainly due to methodological constraints. There is a need for methods, reagents and devices for rapid detection of the cancer cells without the need to enrich a sample.

More generally, rare circulating cells, of which circulating tumor cells (CTC) and circulating stem cells (CSC) are non-limiting examples, are generally thought to represent untapped opportunities for diagnosing and monitoring pathologies and diseases. In the example case of CTCs/CSCs, the cells are assumed to be shed from primary or secondary tumors of patients with advanced cancer and have been detected in the peripheral blood of patients with advanced stages of most types of solid tumor cancers. However, CTCs have also been detected in patients with localized cancers, which may be indicative of increased risk of progression to metastatic disease or very early tumor development. It is possible the rapidly growing pre-malignant lesions shed epithelial cells in sufficient quantity to be captured from the peripheral blood and analyzed for early diagnosis.

Since rare cells are mainly characterized and identified by their morphology and immunostaining pattern, their heterogeneity is a major obstacle for rare cell detection. The rare cells derived from different types of tissues significantly distinguish from each other with different size, shape, and immunophenotyping profile. However, there is broad morphological and immunophenotypical variation within rare cells derived from the same tissue of origin. For example, during epithelial to mesenchymal transition, the expression of epithelial markers on CTCs, such as epithelial cell adhesion molecule (EpCAM) and cytokeratin (CK), may be down-regulated and become undetectable.

Therefore, accurate detection of rare cells based on morphological and immunophenotypical profiling is challenging. Additionally, rare cells may be damaged and fragmented, in vivo and/or in vitro, due to multi-step cell preparation processes, causing inaccurate detection and misinterpretation.

CTCs are characterized as non-leukocytic, nucleated cells that are typically epithelial in origin, and maintain significantly larger diameters than normal blood cells. However, the morphological features of CTCs are now known to be less clearly defined. It is accepted that a significant number of CTCs may lose their epithelial markers and express the phenotypic markers of epithelial-mesenchymal transition (EMT). Subsets of CTCs may represent viable metastatic precursor cells capable of initiating a metastatic lesion. Molecular and phenotypical differences between CTCs and the primary tumor have been documented and may vary by cancer type and disease progression. Additionally, it has been demonstrated that there is heterogeneity among a patient's CTCs. These complexities introduce additional challenges for interpreting CTC analysis results. The analytical methods/assays used will be important to establishing a common set of criteria describing CTCs.

CTC assays may be broken down into three major steps: 1) blood sample preparation and tumor cell separation; 2) cell staining by antibodies or gene probing by DNA probes; and 3) CTC detection. A platform that can characterize the oncogenic alterations in the CTCs may aid in identifying therapeutic sensitivity/resistance which would be critical for early modification of therapeutic regimens contributing to more effective personalized health care. It has recently been suggested that clusters of CTCs may be relatively protected from cell death and that the presence of clusters may be a better marker of metastatic potential than single CTCs. Current enrichment methodologies are likely to disrupt CTC clusters thereby missing these potential indicators of metastatic potential. These enrichment protocols result in a biased capture of the CTCs detecting only those CTCs that conform to the predetermined criteria for capture. For example, the current definition of a circulation tumor cell is that it is cytokeratin positive, has a nucleus, and does not have the leukocyte marker, CD45. After enrichment, the cells are examined for presence of cytokeratin, nuclear staining and the absence of CD45 staining. The problem with this approach is that as the tumor cells progress from the epithelial-like early stage to the mesenchymal-like more aggressive stage, expression of epithelial-like proteins like cytokeratin and the epithelial cell adhesion molecule (EpCAM) is often reduced. Moreover, the more aggressive circulating tumor cells in the sample can be missed using the enrichment approach. Thus, there is a need to overcome the limitations of current techniques of biased enrichment and disruption of CTC clusters to realize the full potential for CTC detection and characterization to positively impact patient outcome.

The antibodies or antigen binding portions thereof used in the example embodiments described herein are coupled/conjugated to a fluorophore molecule, which may be attached directly to an antibody or antigen binding portion thereof to form an immunoconjugate. Immunoconjugates may be formed by direct covalent attachment of the fluorophore to a functional group on the antibody, or the fluorophore may be conjugated to a chelating moiety that is attached to the antibody or fragment thereof. Methods for coupling or conjugating fluorophore to antibodies are known to those skilled in the art.

The fluorescent material (fluorophore) may be any suitable fluorescent marker dye or any other suitable material which will identify the cells of interest. A smear treated in this manner, which may include the blood and/or components of the blood, is prepared and optically analyzed to identify rare cells of the targeted type. For statistical accuracy it is important to obtain as large a number of cells as required for a particular process, in some studies at least ten rare cells should be identified, requiring a sampling of at least ten million cells, and up to fifty million or more, for a one-in-one-million rare cell concentration. Such a blood smear typically occupies an area of about 100 cm². It is to be understood, however, that this is simply one example and other numbers of cells may be required for statistical accuracy for a particular test or study. Other cell identifiers which are being used and investigated are quantum dots and nanoparticle probes. Also, while a rare cell is mentioned as a one-in-one-million cell concentration, this is not intended to be limiting and is only given as an example of the rarity of the cells being sought. The concepts discussed herein are to be understood to be useful in higher or lower levels of cell concentration.

There is a lack of sensitivity in the current state of the art for identifying rare cells, such as circulating tumor cells (OTCs), among a background of 5 billion cells in a milliliter of whole blood. Heretofore, most technologies attempt to overcome this problem by enriching for the rare cells in the blood sample based on predetermined criteria such as size and the presence or absence of certain antigens. The downsides of the so-called enrichment technologies result from the heterogeneity of the rare cells even within the sample from a single patient. The heterogeneity in size and antigen expression in the rare cells limits the efficiency of any enrichment methods.

Various problems associated with rare cell detection are identified and resolved using technology described in in commonly-assigned U.S. provisional patent application No. 62/184,105, filed on Jun. 24, 2015, and in commonly-assigned PCT patent application number, PCT/US2014/071292, filed on Dec. 18, 2014, the contents of both of which are incorporated herein by reference.

One further problem area associated with rare cell detection identified by the inventors is the challenge associated with enumerating the cancer cell load in blood samples. The inventors determined that in real world blood sample slides having cancer cells there are often just a few whole cancer cells to be detected and counted in each sample slide. On the other hand, each blood sample slide includes many cancer cell fragments. These fragments present a dilemma. They can be counted as cells, not counted, or only fragments above a certain size can be counted. Each of these counting approaches may not produce a count that accurately reflects the cancer load in the blood, especially if there are many small fragments. Moreover, if just a few cells counted, then rounding errors may make it difficult to quantify any improvement achieved from cancer therapy between one blood sample test and another.

### SUMMARY

Example embodiments detect rare cells of interest identified using a rare cell detection system. An example rare cell detection system uses a two-stage optical detection process. A first, high-speed, wide-field scan effectively and quickly scans large numbers of cells on a specimen (e.g., slide) to determine the existence of potential rare cells (e.g., cancer tumor cells) that may be only one in every million or so cells investigated. Detection of cells may be based on brightness of a scanned cell relative to a predetermined brightness threshold or threshold range. For example, only the coordinates (e.g., X-Y position) of the cells of interest detected in the first scan that have a brightness that exceeds the predetermined brightness threshold or threshold range may be stored. In example embodiments, the imaged cells in the first stage are ranked based on brightness of each detected fluorophore, size, etc., and their coordinates may be stored in ranked order. A selection is made based on the ranking. In the second stage, coordinates are used to perform more detailed imaging just on those cells at the stored coordinates. For example, for a fixed camera, the specimen (e.g., slide) may be moved on a movable stage to each of the stored coordinate positions. The detailed imaging may be processed in order to improve the accuracy and reliability of rare cell detection. The further imaging and/or processing may include an alert function to alert a human operator or some other machine.

Example embodiments are useful in a system for the detection of rare cells in a population of a large numbers of cells, such as in the range of 1-10 million cells, or even up to 50 million or more cells at a time in a sample T. In tests run with an example prototype rare cell detection system of the type described above that uses a two-step optical detection process, unprecedented speed and accuracy were obtained. The example apparatus/instrument reproducibly identified a single rare (e.g., cancer) cell on a slide containing 10 million white blood cells. The identification of the single rare cell required a scanning process of less than 10 minutes. Rare cells that may be identified by the example embodiments disclosed herein include, without limitation, breast cancer, ovarian cancer, prostate cancer and pancreatic cancer as well as breast cancer stem cells, and diseases other than cancers, tumors, etc.

Detection reagent cocktails may be designed to detect rare cells shed from tumors. Circulating tumor-associated rare cells include cancer cells and stromal cells. Additionally, cocktails may be designed to monitor numerous other conditions, including, but not limited to the detection of endothelial cells shed during myocardial infarction, the detection of activated leukocytes indicating acute inflammation, the detection of bacteria and parasites, the detection of circulating cells expressing viral proteins for monitoring viral infection, and the detection of fetal cells in pregnant women as examples. The leukocytes of people suffering autoimmune diseases or chronic inflammation such as pancreatitis or inflammatory bowel diseases are likely altered compared to those from healthy individuals. Reagent cocktails may be designed to monitor minor alterations in the leukocytes of patients suffering chronic inflammation and autoimmune diseases. A system for the detection of rare cells in these example situations may be used to monitor rare cells and subtle cellular alteration in the circulation system.

Further example embodiments relate to a method and apparatus for detecting the presence of marked cell objects in a sample of cells contained in or on a medium, where there is at least one marked cell object in or on the medium. The term “cell object” includes whole cells and parts or fragments of cells.

Example apparatus and methods for detecting the presence of marked cell objects contained in a sample include an optical system configured to optically scan the sample in a first optical operation during a first time period to generate a first set of image data and data processing circuitry configured to detect, from the first set of image data, marked cell objects in the sample, determine one or more parameters associated with a detected marked cell object, and generate coordinate locations of detected marked cell objects in the sample. The detected marked cell objects include at least a plurality of cell fragments, each of the cell fragments being smaller than a whole cell.

The one or more parameters may include shape, color, intensity, or size.

In one example application, the data processing circuitry is configured to determine cell fragment count information for the sample and to generate output information based on the cell fragment count information. In another example application, the data processing circuitry is configured to aggregate information for detected cell objects for the sample and to output aggregate information as an indicator of progression of disease. Circulating tumor or cancer cell load information may be included in or determined from the aggregate information.

Example embodiments include determining an area associated with each of the plurality of cell fragments, combine the determined cell fragment areas, and divide the combination by a representative whole cell area to generate equivalent cell load information for output. For example, the area of each of the cell fragments may be determined using a fractional-intensity detection measurement of the cell fragment. Similarly, a volume associated with each of the plurality of cell fragments may be determined, the determined cell fragment volumes combined, and the combined cell fragment volumes divided by a representative whole cell volume to generate equivalent cell load information for output. The volume of each of the cell fragments may be determined using a light intensity detection measurement of the cell fragment.

A memory may be coupled to the data processing circuitry and store determined cell object parameter information and coordinate location information associated with the coordinate locations of at least some of the detected marked cell objects. In one example embodiment, the optical system, in a second optical operation during a second time period, obtains image data at the coordinate locations of selected ones of the detected marked cell objects. The data processing circuitry processes the obtained image data to characterize at least some of the selected marked cell objects and generate output information based on the characterization of the selected marked cell objects. Cell fragment count information may be determined for the sample during the first optical operation, and the second optical operation may be selectively performed based on the determined cell fragment count information for the sample. The data processing circuitry may generate thumbnail image files for detected cell fragments for the sample during the first optical operation.

In some embodiments, the data processing circuitry analyzes detected cell fragments for the sample to determine a degree of match between detected cell fragments for the sample and a predetermined cell fragment definition. In some instances, a filtering operation may be used when determining the degree of match. The data processing circuitry may also rank or select certain ones of the detected cell fragments based on how close the detected cell fragments match the predetermined cell fragment definition.

Further embodiments include calibrating the apparatus using a distribution of different uniform size microspheres and to generate statistically-based correction factors for different fragment sizes using scans of the different uniform size microspheres by the optical system. Then, the determined cell fragment count information for the sample may be compensated using the statistically-based correction factors for different fragment sizes.

Example embodiments include methods and apparatus for detecting the presence of marked cell objects contained in a sample. An optical system optically scans the sample in a first optical operation during a first time period to generate a first set of image data. Data processing circuitry detects, from the first set of image data, marked cell objects in the sample and determine one or more parameters associated with a detected marked cell object. The processing circuitry further determines aggregate information for detected marked cell objects for the sample and outputs the determined aggregate information. The determined aggregate information is indicative of progression of disease. One non-limiting example disease is cancer. The data processing circuitry may determine and generate the output information for the sample for different times.

In example applications, the determined aggregate information includes one or more ratios associated with epithelial cell load and metastatic cell load for detected marked cell objects. In other example applications, the determined aggregate information includes an aggregate area associated with the detected marked cell objects for the sample. In other example applications, the determined aggregate information includes an aggregate volume associated with the detected marked cell objects for the sample. In other example applications, the determined aggregate information includes an aggregate brightness value associated with the detected marked cell objects for the sample.

In certain examples, the detected marked cell objects include at least a plurality of cell fragments, each of the cell fragments being smaller than a whole cell. The data processing circuitry is configured to generate and display a histogram of different size cell fragments detected in the sample. The data processing circuitry may also generate and output total cell fragment volume information associated with the sample.

In example embodiments, the data processing circuitry is configured to determine cell fragment count information and whole cell count information for the sample and to generate output information based on the determined cell fragment count information and the determined whole cell count information.

Example embodiments include methods and apparatus to specifically detect the presence of marked cell fragments in a sample of cells contained in or on a medium, where there is at least one marked cell fragment in or on the medium. The sample is optically scanned in a first time period to generate a first set of image data. From the first set of image data cell fragments in the sample are marked or otherwise indicated. At least some of the marked cell fragments are ranked or simply selected, and respective area or volume values for the ranked or selected cell fragments are determined. The respective area or volume values for the ranked or selected cell fragments are combined to generate an equivalent cell count. Information is output reflecting a total cell count for the sample may be based at least in part on the equivalent cell count.

The ranked or selected fragments may include marked cell fragments above a predetermined size.

In example embodiments, from the first set of image data, marked cells in the sample are also detected and counted. The equivalent cell count is combined with a count of the detected marked cells to generate the total cell count for the sample of cells.

In example embodiments, respective area values for the ranked or selected cell fragments are determined, and the combining includes summing those area values and dividing the sum by an area of a representative cell area.

In other example embodiments, respective volume values are estimated for the ranked or selected cell fragments, and the cell fragment volumes are combined into an aggregate cell volume used to generate the equivalent cell count for the fragments. The volume value combining may include summing the cell volumes and dividing by a representative cell volume.

In example embodiments, a computer applies a multiplication factor based on a probability of detection for each fragment size to generate a correction factor. The correction factor may be combined with the equivalent cell count to generate the total cell count.

The optical scan may be performed in a low magnification scan or a high magnification scan.

In example embodiments, a computer generates a histogram of fragments by fragment size.

Although useful in some example embodiments, a representative cell area or volume is not necessary in all embodiments. For example, a representative cell and/or its characteristics may not be known with sufficient certainty, or no whole cell may be available in the sample from which an estimate of a representative cell size may be determined. Moreover, in some instances, a desired parameter characterizing the sample may be a total cell load for the sample, regardless of the size of a representative cell.

Other example embodiments detect the presence of marked cell fragments in a sample of cells contained in or on a medium, where there is at least one marked cell fragment in or on the medium. The sample is optically scanned in a first time period to generate a first set of image data. From the first set of image data, marked cell fragments are marked or indicated in the sample. At least some of the marked cell fragments are ranked or selected, and respective area or volume values for the ranked or selected cell fragments are determined. The respective area or volume values for the ranked or selected cell fragments are combined to generate a total area, total volume, and/or total cell load for the sample. Information reflecting the total area, total volume, and/or total cell load of the cell fragments and/or the total area, total volume, and/or total cell load of the cells is output in any suitable output format including a file, one or more displays of numbers, graphs, bar charts, pie charts, etc. to convey this information to the user, etc.

Further example embodiments detect the presence of marked cell fragments in a sample of cells contained in or on a medium, where there is at least one marked cell fragment in or on the medium. The sample is optically scanned the sample in a first time period to generate a first set of image data. From the first set of image data, marked cell fragments are marked or indicated in the sample with a corresponding brightness or other light value. At least some of the marked cell fragments are ranked or selected based on brightness, size, etc., and the brightness or other light values of all those selected cell fragments is summed into one total value which is indicative of the total amount of detected material in the sample. This total brightness or light level from fragments is recorded and output.

In a further example embodiment, a total area or volume of detected epithelial cells and/or cell load and a total area or volume of detected mesenchymal cells and/or cell load are determined for a sample. Different antibodies and corresponding fluorophores of different colors are attached to identifying constituents within the cells, e.g. cytokeratin or vimentin, that are indicative of each of these stages of CTC evolution, either epithelial or mesenchymal. The protein cytokeratin is associated with an epithelial cell type which are associated with an earlier stage of cancer. The protein vimentin is associated with a mesenchymal cell type which are associated with a later stage of cancer.

A ratio of the total area or volume of epithelial cells and/or cell load to mesenchymal cells and/or cell load detected for the sample is compared, as this may be indicative of the progression of the associated tumor evolution and a ratio of the two is output. The ratio of cytokeratin and vimention, for example, in the detected cells may provide useful information to the diagnostician about the stage of the cancer progression. Cells or cell fragments may be associated with different stages of cancer based on a degree of different fluorescent colors, where a more visible vimentin indication may show that the tumor is at a later stage in its progression. Ratios of these epithelial and mesenchymal cell objects may be indicative of disease progress. Information reflecting the total area or volume of detected epithelial cell objects and/or cell load and a total area or volume of detected mesenchymal cell objects and/or cell load is output in any suitable output format including a file, one or more displays of numbers, graphs, bar charts, pie charts, etc. to convey this information to the user, etc.

The above aspects and example embodiments will be better understood and appreciated in conjunction with the following detailed description taken together with the accompanying figures.

### BRIEF DESCRIPTION OF THE DRAWINGS

FIG. 1 depicts a non-limiting example optical system;

FIG. 2 depicts a non-limiting example implementation of the optical system from FIG. 1 in a housing with an access door;

FIG. 3 shows a flow diagram of a non-limiting example of a rapid, low magnification scan used to initially find candidate points of interest;

FIG. 4 shows a flow diagram of a non-limiting embodiment with example of operations at each low magnification image position;

FIG. 5 shows a flow diagram of a non-limiting example of filtering block;

FIG. 6 shows example raw image intensity data in a 3D plot after low pass filtering;

FIGS. 7A and 7B are flow diagrams for a non-limiting example embodiment related to the first stage process;

FIG. 8 shows an example display of a cell object table;

FIG. 9 is a flow chart diagram illustrating another example embodiment that includes example user histogram interaction before performing the second stage;

FIG. 10 is a flow chart diagram illustrating example steps for the second stage;

FIG. 11 is a diagram illustrating an example of scoring and ranking cell images based on various characteristics associated with images from a cell object table;

FIG. 12 illustrates example computer-implemented procedures for control of the optical system;

FIG. 13 illustrates a flowchart diagram that details another example embodiment of obtaining data from a sample using a two-stage process;

FIGS. 14A-14D show example cell and cell fragments;

FIG. 15 is a flowchart showing example procedures implemented using the computer controller to calibrate the sensitivity of the instrument when detecting small fragments;

FIGS. 16A-16C are example probability graphs obtained when calibrating the instrument and used to correct measured results;

FIGS. 17A and 17B are flowchart diagrams that detail example embodiments of obtaining data from a sample including cell fragments and whole cells using a two-stage parallel process;

FIGS. 18A-18E show examples relating to detecting cell/cell fragment illumination/brightness detection;

FIGS. 19A-19B show example output displays in table form; and

FIG. 20A-20C show further example output displays that illustrate ratios and sizes of detected cell load per sample for epithelial cell load and metastatic cell load in bar graph, curve, and pie chart display formats; and

FIG. 21 shows another example output displays illustrating ratios and sizes of detected cell load per sample for epithelial and metastatic cell load.

### DETAILED DESCRIPTION OF EXAMPLE EMBODIMENTS

The example embodiments disclosed herein relate, in part, to improvements in rare cell detection methods and devices. To facilitate understanding of this disclosure set forth herein, a number of terms are defined below. Generally, the nomenclature used herein and the laboratory procedures in biology, biochemistry, organic chemistry, medicinal chemistry, pharmacology, etc. described herein are generally well known and commonly employed in the art. Unless defined otherwise, all technical and scientific terms used herein generally have the same meaning as commonly understood in the art to which this disclosure belongs. In the event that there is a plurality of definitions for a term used herein, those in this section prevail unless stated otherwise.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “sample” refers to any sample obtained from a subject, including, but not limited to, blood, plasma, broncheoalveolar lavage (BAL) fluid, pleural fluid, fine needle aspirate, cervical smear, tissue, urine, stool, etc.

The terms “label,” “readout-molecule,” “labelling moiety,” “signaling molecule,” and the like, as used herein, refer to agents that are capable of providing a detectable signal, either directly or through interaction with one or more additional members of a signal producing system. Labels that are directly detectable and may be use in accordance with the example embodiments. For example, fluorescent labels, where the wavelength of light absorbed by the fluorophore may generally range from about 300 to about 900 nm, usually from about 400 to about 800 nm, and where the absorbance maximum may typically occur at a wavelength ranging from about 500 to about 800 nm. Specific fluorophores for use in singly labeled primers include: fluorescein, rhodamine, BODIPY, cyanine dyes, 4′, 6-diamidino-2-phenylindole (DAPI) and the like. Radioactive isotopes, such as 35S, 32P, 3H, and the like may also be utilized as labels. Examples of labels that provide a detectable signal through interaction with one or more additional members of a signal producing system include capture moieties that specifically bind to complementary binding pair members, where the complementary binding pair members comprise a directly detectable label moiety, such as a fluorescent moiety as described above. The label should provide a constant and reproducible signal over a given period of time. Capture moieties of interest include ligands (e.g., biotin) where the other member of the signal producing system could be fluorescently labeled streptavidin, and the like.

The term “antibody” as used herein is intended to include, without limitation, whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Nonlimiting examples of such proteolytic and/or recombinant fragments include Fab, F(ab′)2, Fab′, Fv, and single chain antibodies (scFv) containing a V[L] and/or V[H] domain joined by a peptide linker, or mixtures thereof. The scFv's may be covalently or non-covalently linked to form antibodies having two or more binding sites. Polyclonal, monoclonal, or other purified preparations of antibodies and recombinant antibodies may be incorporated or used with the example embodiments. An antibody used for detection of a biomarker, as described herein, may be a labeled antibody. The labeled antibody may comprise a fluorescent label for detection and/or capture of CTC cell surface or cytosolic markers selected, without limitation, from the group consisting of EGFR, HER2, ERCC1, CXCR4, EpCAM, ALCAM, CA125 (Mucin-16), E-Cadherin, Mucin-1, Cytokeratin, PSA, PSMA, RRM1, Androgen Receptor, Estrogen Receptor, Progesterone Receptor, IGF1, cMET, EML4, Leukocyte Associated Receptor (LAR), integrins, Alpha Fetorotein (fetal protein), Alpha Smooth Muscle Actin and the like or mixtures thereof, as further described herein.

Although a microscope slide is described as the substrate of choice for purposes of this discussion, any solid or porous substrate may be employed in accordance with the principles disclosed herein.

Antibody cocktails to detect cells of various types, including circulating tumor cells, have been used before. Cocktails of distinct antibodies employ different fluorophores coupled/conjugated with each type of antibody in the cocktail in order to distinguish one fluorescing signal from the other when used to detect various types of cells in a sample. However, in an example embodiment, a combination of distinct antibodies may be employed in a cocktail using multiple selective and distinct antibodies that are all labeled with the same fluorophore to increase the robustness of the detecting by resonating the fluorescing signal.

Each fluorophore used to process the sample is chosen for a distinctive and separate emission color, and each fluorophore requires a particular wavelength of excitation illumination. Thus, a particular set of optical wavelength filters for the excitation illumination waveband and for the emission waveband is used for each fluorophore. The optical image detected from one such a wavelength combination is referred to herein as a “channel.”

A two stage optical process includes a first, rapid scan stage of taking a series of microphotographs of the sample, typically at low magnification and at high speed. In the following description, a low magnification of 4× is used by way of example only. By using a low magnification, each image field of view is larger which means that the whole area of the sample may be covered quickly in a smaller number of images. For example, if the eventual magnification required is 40×, (a non-limiting example), but the initial scan is done using 4× magnification, the sample area can be covered with one hundredth the number of images. (E.g., one hundred 40× images (at 0.3 mm area each) in a 4× image).

Each scanned image from the sample is searched to find features of interest in accordance with one or more predetermined search criteria, such as brightness above a threshold, size of a group of bright pixels more or less than required, etc., and only some small amount of data regarding identified points of interest in the image data is retained for further investigation and processing. The low magnification image data is preferably discarded, e.g., before the next image is scanned, which can result in a faster search time and less data processing resources being required (e.g., communications bandwidth, computing power, memory, etc.).

Example embodiments use a low initial detection threshold to identify all data points of potential interest in the initial rapid scan. One or more parameter or characteristic values such as coordinate position in the sample, detected brightness in each color, and/or detected size of an object in the image is/are used to describe each of a relatively small group of points of interest. That means much less data storage and data processing are required as compared to saving and processing the huge amount of image data for hundreds of complete images produced in a typical initial optical scan. The parameter value(s) for the identified points of interest may then be further processed, sorted, and selected to identify specific points that will be imaged and analyzed in detail in the second stage. Advantageously, no further first stage scanning of the sample is required even if one or more predetermined detection criteria, e.g., the threshold level for each color channel or the size limits imposed on each object, is changed to modify the list of points of interest. This contrasts with a system where the most likely points of interest are stored from an initial or first scan, and where the entire scan must be reprocessed if the detection criteria change.

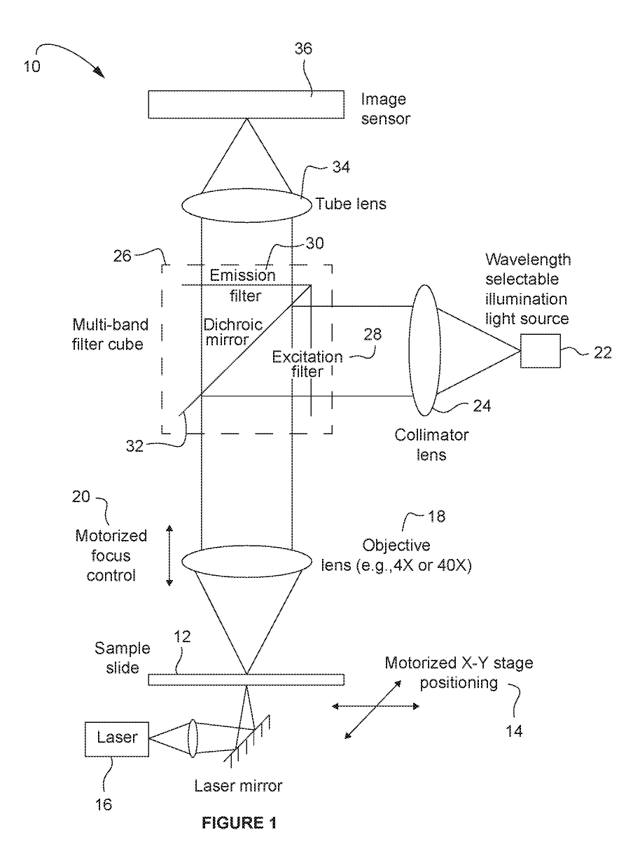

A non-limiting example embodiment of an optical instrument/optical imaging system 10 is shown in FIG. 1. The sample to be searched for rare cells and/or rare cell fragments (both being encompassed by the term cell objects) is on a microscope slide 12 which may be moved in X and Y directions by a motorised stage 14. Above the stage 14 is one or more microscope objective lenses 18, with motorized focus control 20, and elements of an epi-fluorescence microscope including a multi-band fluorescence filter 26 cube, tube lens 34, and an image sensor 36, e.g., a sensitive camera, a time delay integration CCD based sensor, etc.

The filter cube 26 includes a dichroic mirror 32 which passes the fluorescence emission wavelength(s) but reflects the excitation wavelengths. Emission wavelengths are further filtered by an emission filter 30 before passing to the image sensor 36. A wavelength selectable illumination light source 22, such as one or more LED sources, is collimated at lens 24 and filtered by an excitation filter 28 before being reflected towards the sample by the dichroic mirror 32 in the filter cube 26. Different excitation wavelengths may be selected by enabling LEDs of different wavelengths as explained in further detail below.

The objective lens assembly 18 is motorized so that it may be focused by automated control 20, and objectives of different magnification may be selected, e.g., 4× and 40×. Under the slide 12 is mounted a laser 16 that is focussed at the center of the slide 12. The laser 12 may be selectively controlled by an automated control not shown.

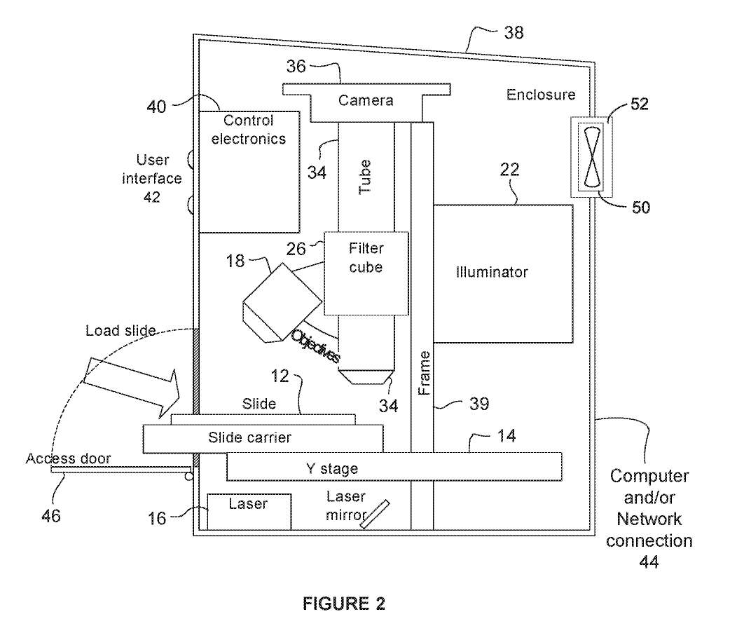

FIG. 2 shows an example implementation of an optical/image processing system provided in a housing 38 with an access door 46 and a frame 39 upon which various elements are mounted. The housing 38 excludes most ambient light and protects the user from intense illumination inside the box. Control electronics 40 are provided to drive motors, activate/deactivate the LEDs and the laser, etc. Optionally, some or all control may be accomplished by one or more computers attached via a data connection such as USB, Ethernet, WiFi, etc. This data connection may also be used to distribute information from the optical system via data communication network(s). Alternatively, all computer control hardware is included within the instrument so that it may operate stand-alone and at likely faster speeds.

Debris may be introduced inside the enclosure when the access door 46 is open. Accordingly, the optical/image processing system may be provided with a fan system 50 and/or a filtration system 52. The fan system 50 and/or a filtration system 52 may be provided in the housing 38 to circulate air into and/or out of the enclosure of the optical/image processing system. The filtration system 52 may include one or more air filters to filter out debris (e.g., dust) from entering the enclosure from outside of the optical/image processing system and to filter the air in the enclosure.

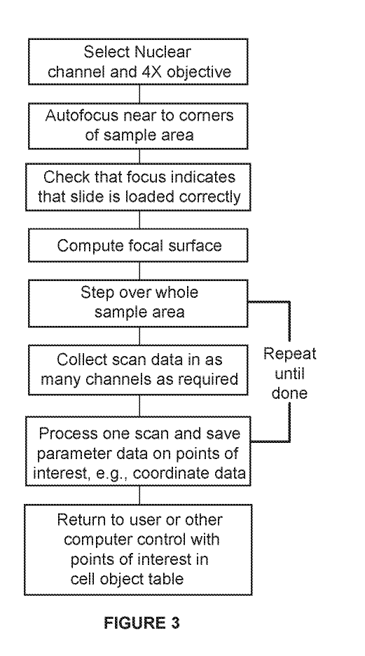

FIG. 3 shows a flow diagram of a non-limiting example of a rapid, low magnification scan used to find initial candidate points of interest. A low magnification objective lens, e.g., 4× magnification, is used to achieve a relatively large field of view and image the whole sample in fewer images. One fluorescence channel is selected for focussing and is referred to hereafter in non-limiting examples as the “nuclear” or “nucleus” channel, referring to a nucleus of a white blood cell. A nuclear stain channel is a good candidate for this focusing channel if the sample is packed with white blood cells, and there are nuclei to be found everywhere in the sample. One or more computers in the instrument then automatically compute the focus surface by taking multiple, e.g., three, focus measurements on the sample, e.g., near corners of the sample.

The computer control also checks that the slide is correctly loaded and not sitting too high by measuring the distance to the slide during the focus measurement. For a correctly positioned slide, this distance is controlled to be within certain bounds to avoid problems when switching to a higher magnification objective with smaller working distance. The computer control determines the focal surface, and controls the optical instrument/imaging system to collect images in one or more channels to cover the sample area. For example, this may be done in a serpentine pattern to minimize travel time between images collected.

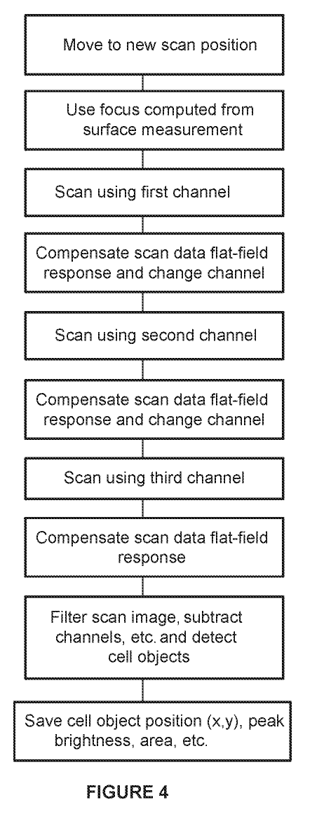

The image sensor scans or steps over the whole same area, and scan data is collected for as many color/wavelength channels as required. For example, for a three channel system, scan data is collected for each of the three different channels for each pixel or point. Each image collected corresponds to a digital image data set that is processed to extract certain parameter or characteristic data, such as a coordinate location on the sample of each local intensity or brightness peak (corresponding to a detected point of interest) detected in the image data set and its measured intensity or brightness in each channel. Advantageously, the large image data set may then be discarded. More details on example scanning procedures are set forth in FIG. 4. This scanning and processing is repeated until the sample is covered. When all images have been collected and processed, the optical system may return control to the user with the data collected for each local peak such as described above. Alternatively, the optical system automatically performs further processing and/or begins the second optical processing stage.

FIG. 4 shows example scanning and processing procedures at each low magnification image position. At a new scan position, the focus to be used is computed by the computer controller from the earlier surface measurements by interpolation. At this position, an image is scanned for a first of one or more wavelength channels, and each image is corrected using compensation data (e.g., flat-field response) previously calculated for this instrument during calibration.

Filtering and processing procedures (described further below) are performed on the image data, e.g., filtering, subtraction, correlation, and/or thresholding of the image data depending on the example embodiment, to detect cells and/or cell fragments, more generally cell objects. Thereafter, and as described above, only a relatively small amount of data, e.g., on the order of kilobytes is saved in memory for each of the possible points of interest including each point's coordinate location on the sample and possibly one or more parameters such as peak brightness, area, etc. The image data, which can be quite large, e.g., on the order of many gigabytes, is then discarded.

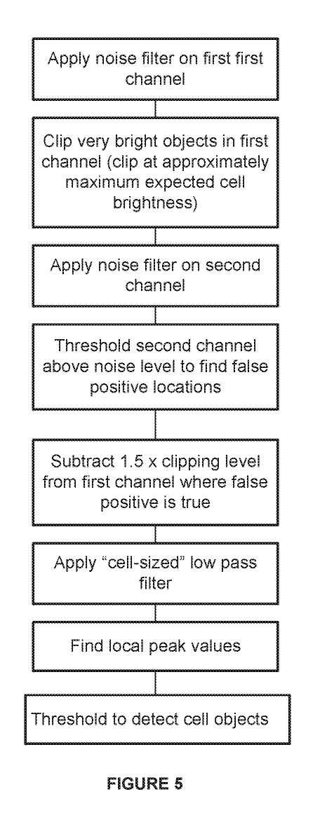

The flow diagram of FIG. 5 shows procedures for example embodiments that filter and process the saved parameter data, such as likely target cell object coordinates, intensity values, etc., associated with each channel at each point to reduce noise, remove false positives, detect local peak values, and perform thresholding. First, the saved parameter data on the first channel (the data relating possible candidate target cells) is filtered with a noise filter to reduce noise. This may include for example filtering using a median filter to reduce salt and pepper noise and other low pass filtering.

In example embodiments, two or more wavelength/color channels (i.e., images captured with different wavelength filter sets corresponding to different fluorophores) are captured in the scan for advanced processing to remove false positive locations. One wavelength/color channel corresponds to potential target whole or fragment cells, and a second wavelength/color channel corresponds to false positives. False positive locations are locations in the scan that falsely present the characteristics of a targeted cell objects, but on further qualification, may be found not to be a targeted cell object. Very bright objects that are brighter than typical for the target cells to be found in the channel representing these objects (referred to as the first channel or sometimes as the “peak channel”) are clipped at a clipping-level just above an expected intensity level. These very bright objects may be debris. Therefore, the filtered first channel intensity or brightness data is then preferably clipped (to reduce the dynamic range required to process intensity or brightness data) since false positive intensity or brightness data is often much greater than target cell intensity or brightness data.

A second fluorophore is used to mark false positive objects such as damaged white blood cells as well as debris that may be less specific in their stain uptake. A noise filter is applied to the second channel (associated with false positives) parameter data, and a simple threshold is used to detect responses in the channel used to read this false positive fluorophore. Wherever a pixel brightness above that threshold is detected in the second channel image, i.e., a false positive is detected, a value greater than the clipping-level (for example, 1.5 times the clipping-level) is subtracted from the corresponding the first channel intensity data (e.g., multiply false positive intensity by 1.5 times the clipping level) to produce a negative peak thereby effectively removing the false positive as a target candidate.

Multiple wavelength/color channels may be combined and processed in the digitized analog domain to retain more information about brightness and shape than a system that thresholds each channel first and performs a digital gating function between multiple channels. Peak height, peak location, and/or area (number of adjoining pixels) above a threshold are examples of parameter information that may be retained from each image for each wavelength/color channel scanned in the low magnification scan. This parameter information may later be used for sorting points and in making selection decisions prior to the second optical stage operations.

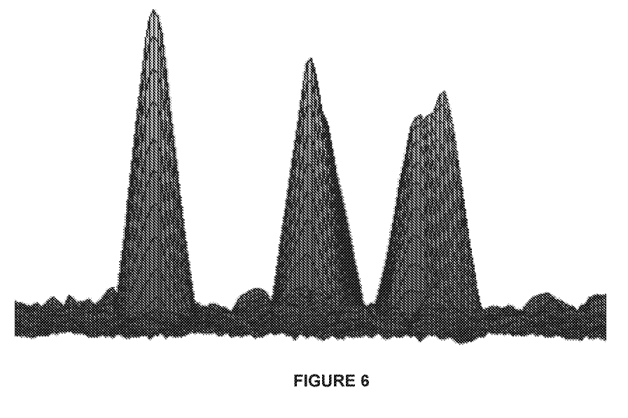

The remaining image data for the first channel is then low pass filtered using a cell-sized or cell fragment-sized low pass filter to identify local peak values in the image data. The low pass filter may be implemented, for example, as a two-dimensional finite impulse response filter, of approximately similar size to a cell or cell fragment so that each cell or cell fragment becomes a point spread function in the filtered image. The point spread function has the advantage that it has a single (thus unambiguous) local peak response that is approximately in the center of the object for objects that are of approximately the filtered size. A filter sized to a smallest target cell ensures that small cells are not lost. A cell object filter may for example be a unit circular filter, or Gaussian filter, of approximately the size of a target cell, or the size of a smaller target cell object if there is an expected range of sizes. FIG. 6 shows an example after low pass filtering.

After low pass filtering, the magnitude of the cell object brightness or intensity is then reflected in the height of the local peak from the filter output, and the centroid of the cell is approximately at the local peak. The locations of centers found using local peaks are less ambiguous than centers found from the areas of threshold detected pixels, which may form irregular shapes and may comprise clusters of cell objects with more than one center. The determination of cell object centers using local peaks also facilitates the identification of duplicate objects, which may have overlapping images.

Having found local peaks, a threshold is then applied to the local peak heights to detect only points or objects meeting a brightness threshold parameter (that is above the noise level) or other parameter(s). In this way, filtered objects are more easily distinguished from noise and thresholding helps to identify points of interest.

An appropriate detection threshold is typically a function of the sample preparation, and in particular example embodiments, the concentration (e.g., fluorescent brightness) of the staining agents. It may not be easy or even possible to retain absolute repeatability in the sample preparation or between samples, so a desired threshold may vary from sample to sample.

Example embodiments use peak heights to locate rare cell objects. In other words, instead of using a simple threshold detection, example embodiments use the local peak height to detect likely cell objects during the scan. Furthermore, where clusters of cell objects are partially merged in the image, their local peaks may still be distinct and separate leading to more accurate cell object counts. These peak coordinate positions and peak heights are saved in memory before threshold detection (which can be done after the scan is completed, as described above). The coordinate location of/positional information from the local peaks is more readily interpreted for locating and counting cell objects, including cell objects that are partially merged in the image.

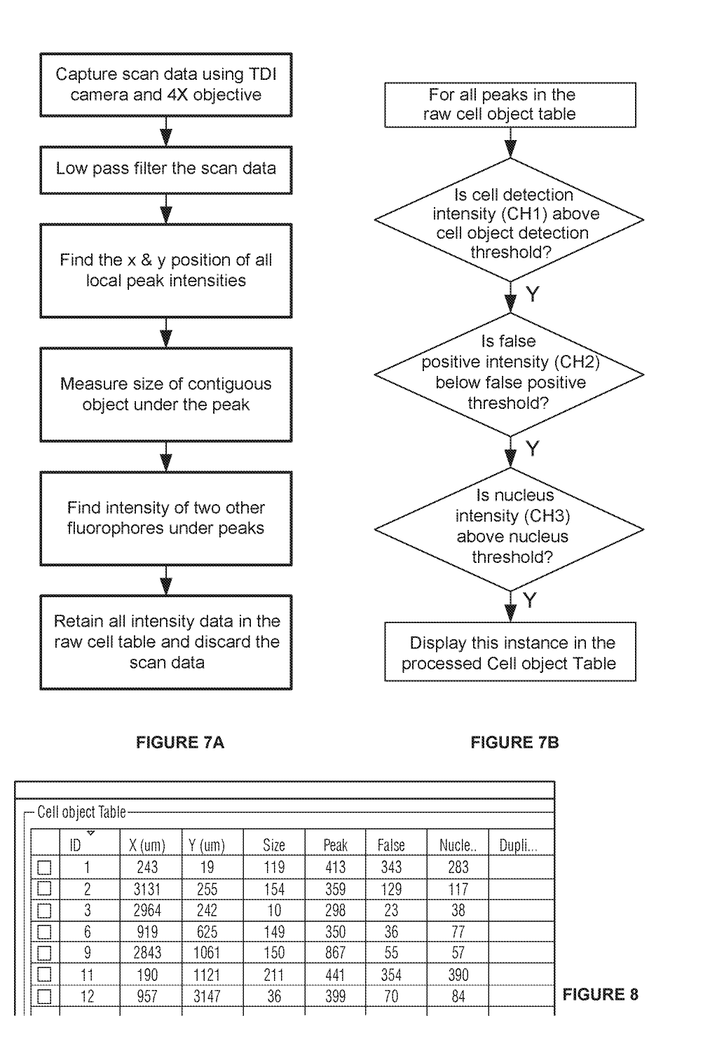

FIG. 7A is a flowchart diagram that details an example embodiment of processes to fill a cell object table after low resolution image collection. The scan data is captured using an image sensor, e.g., a TDI camera, and a low magnification objective lens, e.g., 4×. The scan data is low pass filtered, and the x & y position of all local peak intensities are determined. The size of contiguous object(s) under the peak is measured, and the intensity of an optional second channel (false positives) and an optional third channel (nuclei for autofocus) is/are determined. Parameter data such as the intensity and coordinate data is saved in a raw cell object table and the scan image data is discarded.

FIG. 7B is a flowchart diagram that details an example embodiment of processes for filtering the raw cell object table data. For the local peaks in the raw cell object table, a determination is made whether any cell detection intensity for the first channel is above a cell detection threshold. For the second channel, a process is performed to identify any false positive intensity data below a false positive threshold. For the third channel, a process is performed to identify any nucleus intensity data above a nucleus threshold. The results are entered into and displayed in a processed cell table.

An example cell table is shown in FIG. 8 showing point/pixel identifier, X & Y coordinate location of the intensity peaks found in the low resolutions for the cell object detection fluorophore, i.e., the peak (CH1) intensity. The cell object table also includes the area of the object under each peak, i.e., size, and the intensity of two other fluorophores indicating false positive (CH2) and presence of a nucleus (CH3) under the same peaks. Also indicated is the likelihood of a duplicate or co-located occurrence being found. The displayed contents of the cell object table includes only those instances that remain after filtering with the histogram thresholds.

Sometimes duplicate instances of the same cell object may be found, e.g., because the cell's image was split into two parts due to pixel noise, image overlap, or the same cell object is found near the edges of two adjacent images of the scan. The problem of finding duplicate instances because of fragmentation of a cell object image was discussed above. This can often be solved by low pass filtering so that the cell object is represented not by a collection of pixels from the camera but by pixels assembled into a point spread function, where the local peak of the point spread function is nominally the center of the cell object. Because a large sample is typically scanned using several images there is also a possibility of the same cell object occurring on the edge of one image and on the adjoining image. If low pass filtering is used a small range of pixels contribute to each filtered pixel and so properly filtered results cannot be achieved right up to the edge of the image. In that case slightly overlapping images may need to be collected so that the unfiltered border around each image can be discarded. By finding local peaks the cell objects found near the edge of one image should align exactly with those in the next image but there may be errors due to image distortion, etc. In this case a computer-implemented algorithm is required to discard duplicates. Suspected duplicates are those cell objects found within a small radius of one another, and these are marked as such in the cell object table so that they may be viewed and discarded if required or desired. In a large field of view, e.g. a 4 megapixel camera the overlap required for low pass filtering is a small part of the total image area, typically less than 1%, and so the duplicate problem is small.

In example embodiments, a histogram of local peak heights is created, and decision thresholds may then be set, e.g., interactively by a user or using a computer program, on or using this histogram to bracket the peak heights that may most likely represent the cells of interest. A histogram of peak heights is advantageous as compared to an image brightness histogram. One bright peak in an image will fill the brightness histogram up to and including the peak height. When several peaks are in the image, it is difficult to discern their distribution of heights. However, using a histogram that only contains peak heights, each peak contributes only one value to the histogram at its peak height, and therefore, clusters of similar heights that may represent the cell object brightnesses are readily discerned, and in a well prepared sample, there may be a clear space below this cluster which becomes an optimal position to place the detection threshold. Similarly, bright debris may produce some higher peaks, but unlike a brightness image histogram, these debris peaks do not smear lower values in the peak height histogram which would have obscured the information of real interest in a brightness histogram.

The histogram of local peak values contains significant useful information. For example, the slide quality may be scored. In one scoring example, a clear separation of clusters in a histogram may be taken as an indication of the preparation cleanliness and also yield a confidence factor for the results. The shape may readily be analysed by a computer to determine this quality, and also where to put the thresholds specifically for each sample slide. Fully automated, unsupervised operation is thus possible including slides being fed from an automatic slide carousel. The sum of the histogram frequencies between the thresholds is a direct estimate of the number of cell objects found on the slide, and in some instances, it may be that this is the complete and only result required from the test. There is some potential for duplicates and for clusters that are not fully resolved for which a statistical allowance may be made.

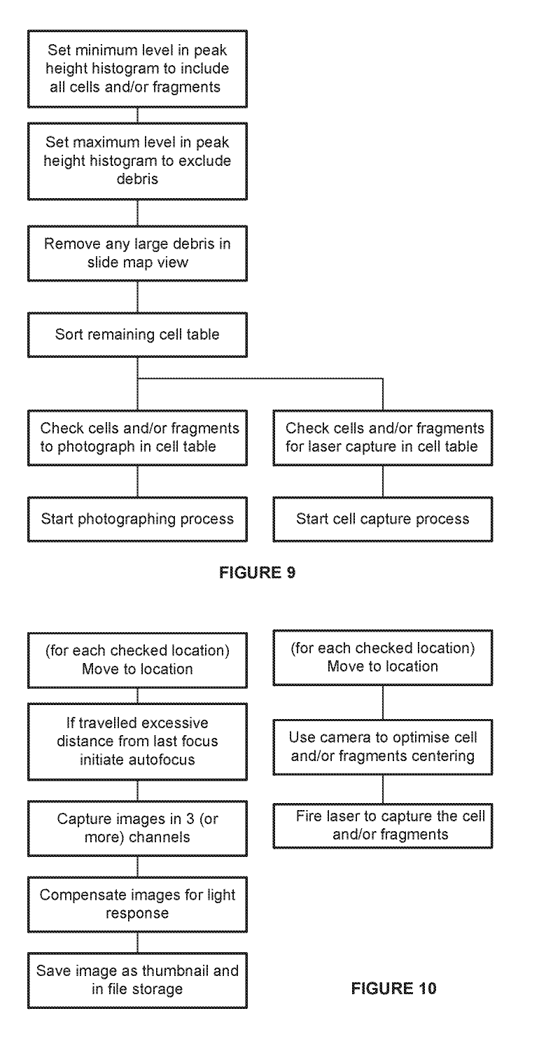

In example embodiments, a user may interact with the cell object table, cell object map, and/or histogram. A flow diagram of example interactions is shown in FIG. 9. A minimum level in peak height of the histogram is set to initially include all peaks. A maximum level in peak height in the histogram may be set to exclude debris. Areas may be selected for removal, and the corresponding items are also removed from the cell table. Remaining entries in the cell object table may then be sorted based on one or more suitable criteria such as intensity, and cells are selected for stage 2 processing such as individual micro-photography or for targeting with the laser for cell recovery.

To collect microphotographs, the stage is moved to each position. For laser capture, the laser 16 is directed on to the cell object. This may require more precision than can be achieved from the cell object position recorded in the cell object table. In order to accomplish fine adjustment of position, control software programs executed by one or more computers will optically position the target cell object in the center of the field of view before the laser is activated.

FIG. 10 is a flow diagram illustrating example procedures for stage two optical processing. For each selected location in the cell table, the image sensor and/or laser is moved to the cell object location. Autofocus may be required for the image sensor, and the image sensor may be used to optimize cell object centering for laser capture. The laser is then fired to capture the cell object, i.e., extract that cell object from the slide for further detailed examination and/or testing of that cell object as described above. On the imaging path, images are captured for multiple channels (three are used in this non-limiting example). The image is then saved. Example embodiments begin the stage two process immediately after the stage one process is completed, but before the final selection of the cell objects to be photographed is made, to further speed up the overall process.

To collect microphotographs, the stage is moved to each position. A high quality focus adjustment is required to image the cell objects but autofocus for every image can add considerably to the process time. A fast auto focus method has been developed to reduce processing time.

FIG. 11 is a diagram showing further processes that may be performed in the stage two optical processing including scoring characteristics against user defined limits by image processing for each high resolution cell object image. A combined score based on multiple test characteristics such as the examples shown in FIG. 11 may be used to rank or select cell object images. Example test characteristics include cell object size, cell object roundness, cell object brightness, nucleus brightness, nucleus size, whether the cell objection is part of a cluster, and/or false positive indications.

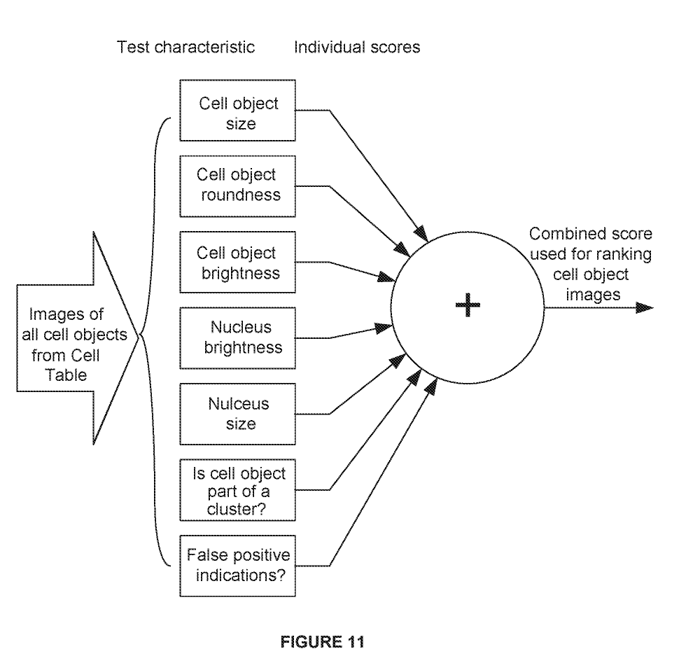

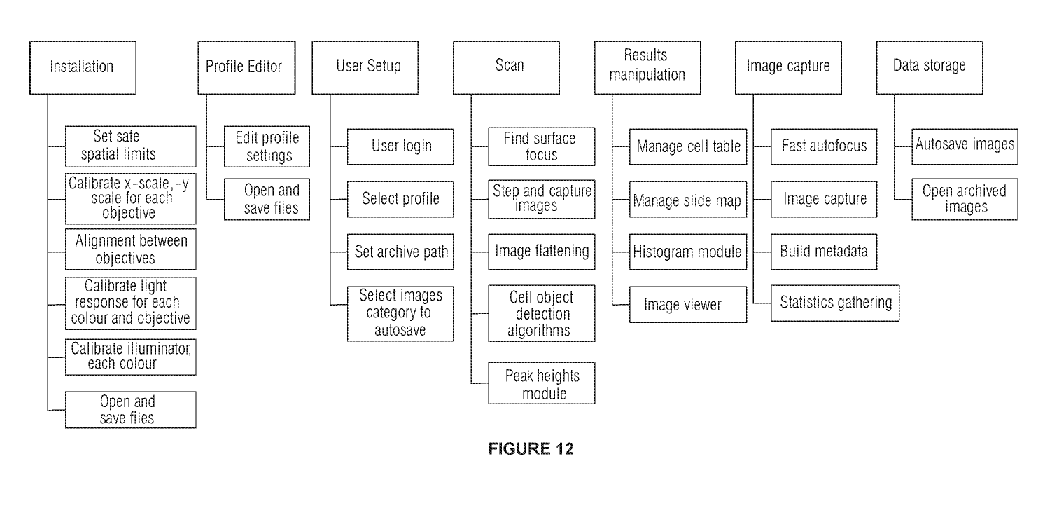

FIG. 12 shows example functions in the optical instrument/system control performed using one or more programmed computers. These are grouped under several main headings that correspond to example phases of operation.

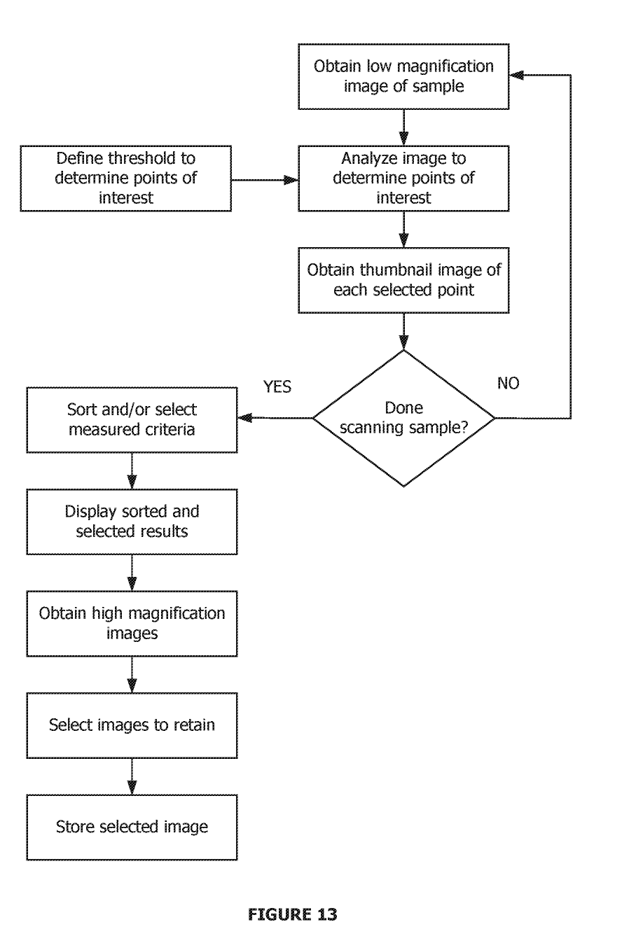

FIG. 13 illustrates a flowchart diagram that details another example embodiment of obtaining data from a sample using a two-stage process. One or more operations of the example embodiment may be performed by one or more hardware data processors associated with the optical system(s) described in this application. FIG. 13 illustrates a two stage process that may use a rapid scan of the sample to find points of interest by one or more criteria that can be observed at lower magnification, and after reducing the extent of the required search, a second stage may be performed just on selected points of interest in more detail.

A low magnification image may be obtained and analyzed to determine points of interest. The points of interest may reveal potential locations of rare cell objects. The points of interest may be determined using one or more criteria (e.g., brightness of point above a threshold). The located points of interest may be ranked based on the one or more criteria. A predefined number of points of interest (e.g., top ranked points of interest) may be selected for further analysis. The predefined number of top ranked points may be pre-set or selected by a user. For example, the predefined number of points may be set to provide 5,000 or 10,000 top ranked points of interest for further analysis. While several hundred points of interest may be sufficient to return points of interest that correspond to a desired rare cell object, setting the value higher (e.g., 5,000 or 10,000) may ensure that the selected points of interest will include all wanted points of interest.

The threshold(s) of the one or more criteria may be adjusted to control the number of points of interest that are provided for each of the low magnification image. The threshold may be set manually by a user, may be a pre-set value set in advance of the scan, and/or may be adjusted automatically. The one or more hardware data processors may receive a user input setting the threshold or retrieve the threshold value stored in storage. It may be beneficial to set the threshold to a value that keeps enough data from which to sort and find the desired rare cell object, but equally the threshold should be set to a value that is not so low that a vast amount of data is kept, overflowing available storage and/or unduly compromising rapid pre-selection.

At each point of interest in the low magnification image, thumbnail images may be obtained and stored in storage. The thumbnail image may be a sub-image of the low magnification image. The thumbnail may be associated with other information about the points of interest (e.g., point coordinates, peak intensity, approximate size, and/or intensity of two other wavelengths at same coordinates). While other example embodiments may store only limited information (e.g., peak intensity, approximate size, and/or intensity of two other wavelengths at same coordinates) for each point of interest, this example embodiment may also store a thumbnail image corresponding to each of the points of interest. The thumbnail images are associated with the corresponding point of interest. In some embodiments, a single thumbnail image may correspond to a plurality of points of interest if the points of interest are clustered together and the single thumbnail image captures the clustered points of interest.

While more storage may be needed to store the thumbnail image (e.g., 200 megabytes from a low magnification scan), the thumbnail images, which may be on the order of 10,000 for a sample, may be quickly post processed, sorted and selected, either by a computer or visually by a user, to find any images with potential rare cells or identify images that should not be further processed. In addition, the amount of storage used for the thumbnail images is still significantly less than used by traditional methods using a single scan. As discussed in more detail below, the thumbnail images may allow the sorting or selection criteria to be changed interactively after the first scan without having to perform additional scans of the same area of the sample. Thus, the time needed to perform the first and second stages for a sample, and particularly for a sample of which content is unknown, is reduced.

Accordingly, the thumbnail image provides information that can be quickly reviewed by a user to easily disqualify a point of interest as being a potential rare cell object or select points for further processing. For example, a thumbnail image with debris (e.g., dust) on the sample that is selected by the computer as being a point of interest (or a cluster of points of interest) may be quickly visually reviewed by a user to determine that the thumbnail image and the point of interest associated with the image should not be further analyzed because it contains debris and does not contain a potential rare cell.

The process of obtaining low magnification images, selecting points of interest and obtaining a thumbnail image at each selected point of interest may be repeated until the whole sample is analyzed. These steps do not have to be completed in the order shown in FIG. 13. For example, in one embodiment, before the points of interest are selected, all of the low magnification images may be obtained. The threshold may be defined by a user input before the scan of the low magnification images is started and/or may be adjusted while the low magnification images are obtained.

The resulting points of interest and low magnitude thumbnail images may be sorted and measured criteria selected. The sorting may sort the selected points of interest based on the one or more criteria. In one embodiment, the one or more hardware processors may receive user inputs selecting the criteria by which to sort the points of interest and/or the thumbnail images. In another embodiment, the one or more hardware processors may automatically sort the results based on pre-defined criteria and the user inputs may modify the criteria for sorting the obtained data. For example, the points of interest may be sorted or selected automatically based on the brightness levels of the points and/or a user input may modify the sorting or selecting criteria to sort the results based on the diameter of the points of interest.

The sorting and/or selection of the measured criteria may be performed using a table and/or histogram of points of interest. For example, a histogram of the selected points of interest may be created and displayed. A different thumbnail image may correspond to each point ranked in the histogram. Based on the displayed histogram a user may set a new minimum threshold to remove some of the detected points of interest, set a maximum threshold to remove some of the points of interest, and/or identify false positives to remove some of the points of interest. The display of the histogram may include displaying a thumbnail image when a user input selects a point on the histogram. During the sorting and selection of the measured criteria user inputs may remove one or more points of interest to remove false positives, noise, and/or excessively bright data (e.g., due to presence of dust on the sample).

The results of the sorting and selection of the measured criteria may be displayed on a display coupled to the one or more processors. The results may be displayed in a table with each entry of a point of interest being provided with information about the point of interest (e.g., coordinates or intensity level) and/or the thumbnail image of the point of interest. The thumbnail images may be displayed in a linear sequence (e.g., as a filmstrip) so that they can be quickly visually inspected. The sequence of the thumbnails may be updated if the criteria used to sort the points of interest is changed and/or points of interest are removed from the table. The user may be provided with a user interface to select which information is displayed with each entry in the table.

A high magnification image may be obtained at each of the remaining points of interest. The stage may be moved such that the high magnification image may be obtained at the coordinates of the point of interest. The high magnification image may be obtained with higher magnification objective as compared to the low magnification image. For example, the higher magnification objective may be a 40× objective lens.

The obtained high magnification images may be displayed on the display and one or more user inputs may select the images to be retained. In one embodiment, the high magnification images may be displayed along with the corresponding low magnification thumbnail images and/or other information about the corresponding point of interest so that the user may make a more informed selection of the images with the rare cell objects. In another embodiment, user inputs may select which high magnification images should be discarded.

The selected images may be stored in the storage coupled to the one or more processors and/or transmitted (e.g., over a network) to another storage. The selected high magnification images may be stored in association with the points of interest and/or the thumbnail images.

As explained in the introduction, a problem area associated with rare cell detection identified by the inventors is the challenge associated with enumerating the cancer cell load in blood samples. Blood sample slides often contain just a few whole cancer cells but many cancer cell fragments. There are multiple counting approaches for fragments such as: (i) count fragments as cells, (ii) do not count fragments as cells, or (iii) only count fragments above a certain size. But these fragment count approaches may not produce counts that accurately reflect the cancer cell load in the sample, especially if there are many small fragments. Moreover, if only a few cells are counted for a scanned sample, then rounding errors may make it difficult to quantify any improvement achieved from cancer therapy between one blood sample test and another.

A better approach is to detect as many fragments as possible and aggregate them into an equivalent whole-cell count that more accurately reflects the cancer cell load. This equivalent whole-cell count is typically more accurate than a simple count of fragments over a certain size and also often results in a non-integer value which may provide more resolution and precision in the measured value, especially for very small total cell loads.

In an example embodiment, all fragments are detected in a slide sample and their respective areas measured. These areas are summed and then divided by the area of a typical whole cell. The typical whole cell area may, for example, have been determined in advance for the kind of cancer expected and this area pre-programmed into the instrument. This may be necessary in the case that there are no whole cells in the sample for reference. If there are whole cells then the instrument may for example be programmed to automatically measure and use the area of the highest ranked whole cell found in the same scan.

In another example embodiment, fragments are viewed as part of a 3-dimensional cell. The volume of each fragment is estimated or otherwise determined, and the fragment volumes are reassembled into an equivalent number of whole cells. Very small fragments may weigh less heavily in their contribution to an aggregate cell volume, and so in some example implementations, it may be acceptable to ignore the smallest fragments.

The equivalent cell count process may in example embodiments be performed using results from the first stage, low magnification scan. This eliminates the need to image many small fragments individually at high magnification, a time consuming process. Instead, high magnification imaging is limited to the largest fragments and whole cells for the purpose of identifying the nature of the cells.

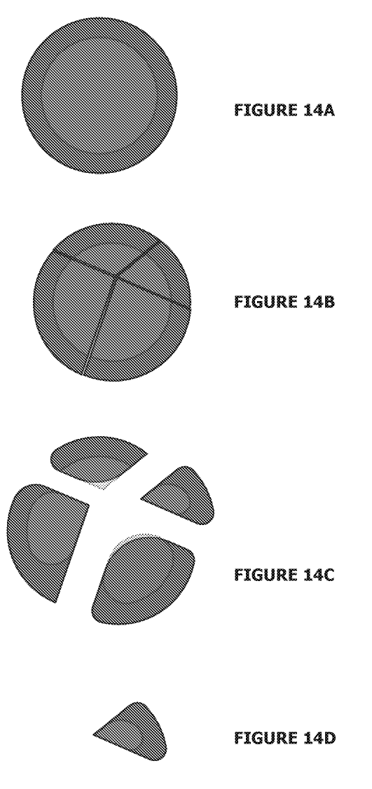

FIG. 14A shows a whole cell, and FIGS. 14B-14D show diagrams representing steps to cell fragmentation and one typical resulting fragment in FIG. 14D. The fragments may be large or small, but likely there is both cytokeratin (CK) and nucleus material in most fragments. FIG. 14D shows just one fragment, which might not generally be recognized and counted as a rare cell due to its small size and because it may be of a more irregular shape. Although small, this cell fragment has cytoplasm material that has taken up the fluorocancer marker and nucleus material that has taken up the nucleus marker. Although too small to be an intact rare cell, this fragment with both markers present is part of a rare cell. Instead of counting it as a cell, or not, its area and/or volume is then integrated with other fragments to contribute, in proportion to its size, towards the measured total rare cell load which is indicative of disease progress.

Rather than attempting to piece such cell fragments together into their original cell form, the computer controller is programmed to calculate an estimate of the number of whole cells that have fragmented. If fragmentation occurred as in FIGS. 14B-14D, then the respective areas of all the fragments detected are added together to arrive at an equivalent number of cells that must have fragmented to create these fragments as described in one example embodiment above. However, given that the cells are 3-dimensional objects and fragmentation could occur in any plane, a preferred but still example approach, as also described in one example embodiment above, is to estimate the area and/or volume of the fragments as a fraction of a typical intact rare cell volume, and sum these volumes to arrive at a total equivalent rare cell load. From a two-dimensional cell fragment image, the depth of each fragment cannot be detected, and therefore, an estimated or averaged cell fragment depth is determined.

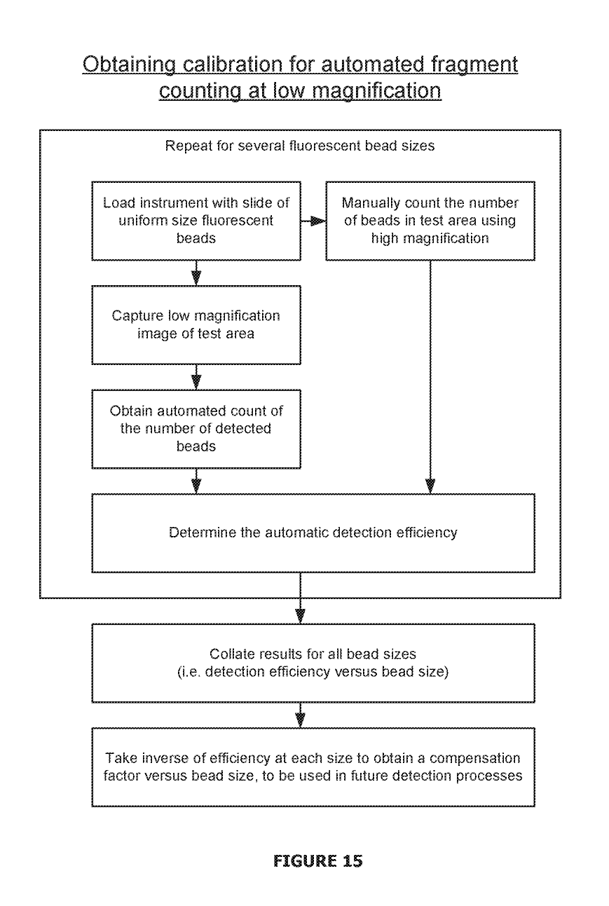

FIG. 15 is a flowchart showing example procedures implemented using the computer controller to calibrate the instrument by finding its efficiency in detecting fragments of different sizes. The instrument is loaded with one slide at a time having a distribution of a particular uniform size of small fluorescent beads or microspheres (i.e., small spherical particles) with diameters in the micrometer range (typically 1 μm to 10 μm). The instrument images the fluorescent beads a low magnification objective during scanning, and the instrument is instructed to automatically count the number of beads detected in the image. A high magnification objective is also used to count the actual number of beads in the same area, e.g., manually. The ratio of these two bead counts gives the detection efficiency of the automated count using the instrument's low magnification objective for one particular small particle size. The process is repeated using uniform sized fluorescent beads of several different sizes, (e.g., 1 μm, 2 μm, 3 μm, etc.), to determine the instrument's automatic detection efficiency versus bead size. The inverse of this efficiency for each particle size is a correction factor that can be used to apply a statistical compensation to future automated counts of small particles such as rare cell fragments using the low magnification scan, which is advantageous because it is much faster than counting using the high magnification scan.

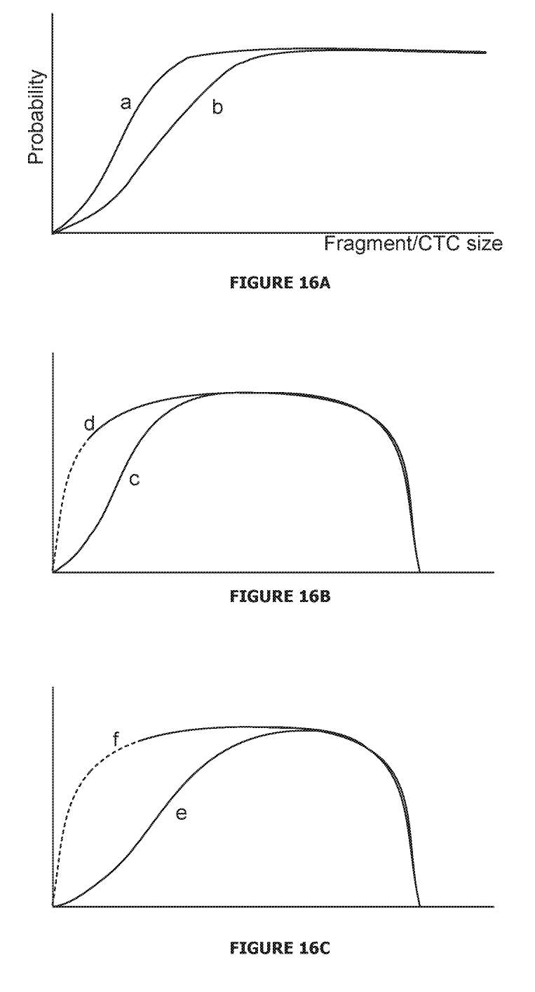

FIGS. 16A-16C are example probability of fragment detection graphs for different fragment sizes. In these plots the horizontal axis indicates fragment size from zero up to approximately a nominal full cell, and the vertical axis is probability of detection versus size in FIG. 16A, or the number of fragments versus size in FIGS. 16B and 16C. It is expected to have close to 100% probability for detecting larger cell fragments or full cells, i.e., the upper flat portion of the curve, but the detection probability falls off for smaller size fragments. At a very small size of fragment, e.g., approximately the pixel size of the camera or the resolution limit of the optics, detection may be quite difficult. FIG. 16A shows curve a for a higher magnification where it is easier to detect small fragments. Curve b in FIG. 16A shows a lower magnification where small fragments that approach the size of one camera pixel are harder to detect.

FIG. 16B shows the results of fragments found from a sample scanned at a high magnification detecting nearly all of the fragments which are ordered by size to produce a distribution similar to curve c. The number of very small fragments detected is small. Dividing the distribution curve c by the probability of detection curve a corrects the number of smaller fragments found by the probability of detecting them and provides a new distribution of fragments as shown in curve d. So for example, if the actual number if fragments on the sample that may be measured as 1 um in diameter is 100, but the probability of detecting fragments of this size using this instrument is 50%, then it is likely that approximately 50 fragments of this size will be detected. If the probability of detection for fragments of this size is known in advance by calibrating the instrument as described in conjunction with the example calibration procedure shown in FIG. 15, then the measured number of approximately 50 fragments can be divided by the probability of detection (a statistical correction or compensation factor) to arrive at a more correct figure of approximately 100. After a similar associated statistical correction or compensation factor is applied for each desired fragment sizes, the distribution curve d more accurately represents the distribution of fragments in the sample for smaller fragments and improves the estimate of the total fragment distribution in the sample.

For the very smallest fragments, the correction may be less helpful because correcting very little measured data by a large correction factor amplifies any noise, which is undesirable. As a result, in one example implementation, corrected fragment detection results are used only down to a predetermined lower bound which excludes the region of the very smallest fragments for which the probability of detection is low and there is insufficient measured data, e.g., as shown by the dotted line in curve d. Fortunately, the inventors recognized that the very smallest fragments are relatively insignificant. A spherical fragment that is one-tenth the diameter of the original cell has a volume of one thousandth of the cell, so discarding this fragment has little impact on the total volume determined by summing all other fragments and cells detected. Thus, removing the smallest part of the measured and corrected distribution, i.e., removing the portion in curve d, often produces an acceptable overall result.

This statistical correction approach for high quality images is advantageously applied to lower resolution images obtained during the first scan stage. Analyzing for fragments in example scan images provides a distribution (e) shown in FIG. 16C to which correction is applied using the known probability of identifying fragments at low resolution (such as curve (b)). The correction produces a corrected distribution (f) in FIG. 16C which is similar to the corrected distribution using high resolution images shown in curve (d) in FIG. 16B. One difference is the uncertain result region shown by the dotted part of curve (f) is larger than the uncertain region in curve (d). Even so, if this uncertain region is statistically insignificant, i.e., the uncertain particles would not contribute significantly one way of the other, then the result is valid and is produced much faster and more efficiently.

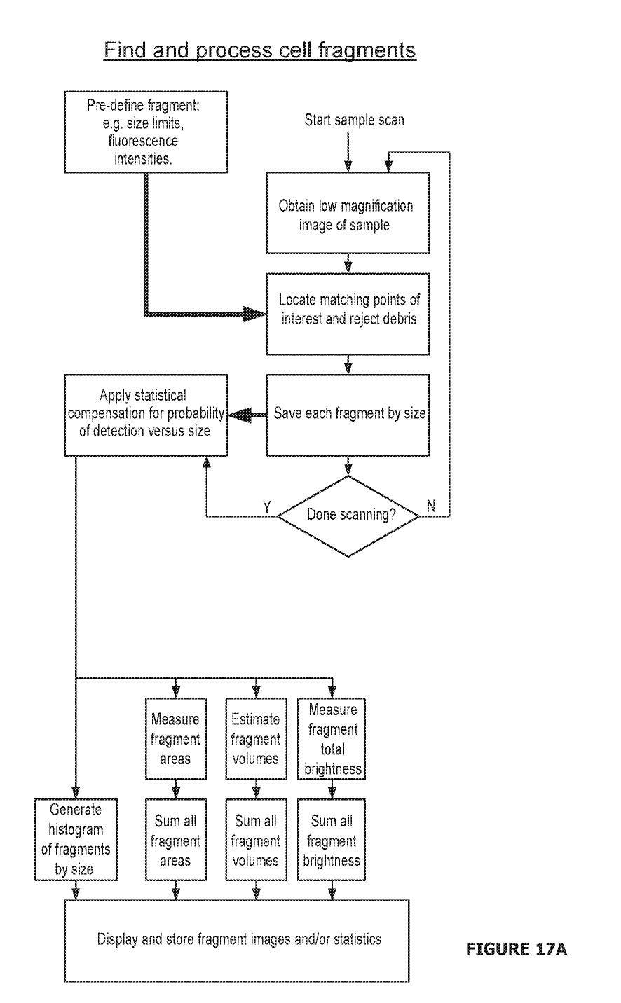

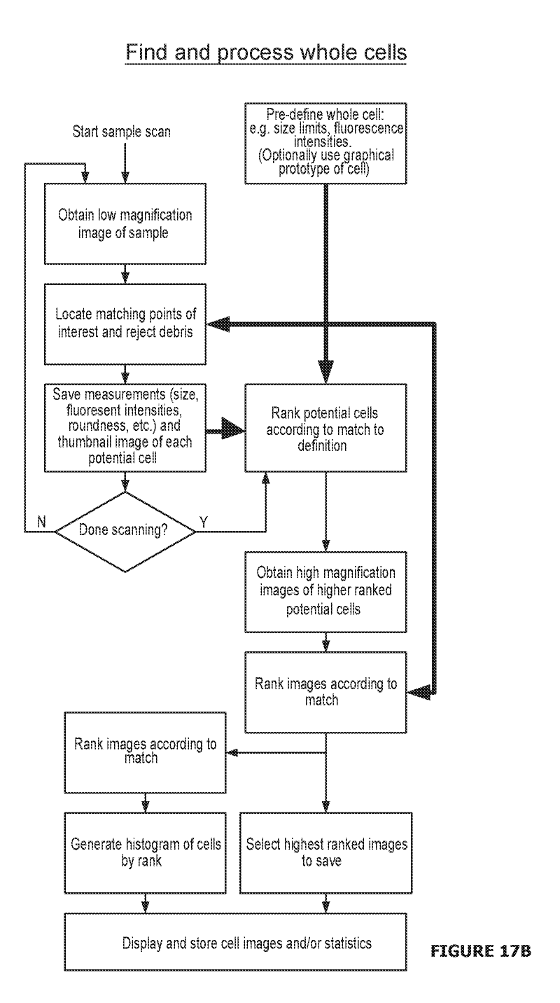

FIGS. 17A and 17B are flowcharts that detail example steps to obtain data from a sample using parallel two-stage processes to identify and process cell fragments and full cells in that sample. The steps shown do not have to be completed in the order shown.

One or more operations of the example embodiment may be performed by one or more processors (e.g., the computer controller) associated with the optical system described in this application. The two stage process applies to both FIG. 17A which shows the steps to identify cell fragments in the sample and to summarize the data, and to FIG. 17B which shows the corresponding steps to identify full or complete cells in the sample and to summarize that data. Each process in FIGS. 17A and 17B uses a first stage rapid scan of the sample to find points of interest by one or more criterion that can be observed at lower magnification, and after reducing the scope of the required search, a second stage may be performed just on the selected points of interest in more detail. While two processes are shown separately in FIGS. 17A and 17B, it will be understood that many of the same steps are performed in FIGS. 17A and 17B and that the processes can be run in parallel so that the common steps need only be performed once for each pass.

This example embodiment includes using a characterization of the target cells and cell fragments to perform the two stage process. The definition of a cell and of a cell fragment may be used to identify potential rare cells and rare cell fragments, find points of interest in the sample from low magnification images and/or high magnification images, rank and/or otherwise select points of interest, rank and/or otherwise select thumbnail images, and/or rank and/or otherwise select high magnification images. For example the distinction between points of interest to be processed as either cell fragments or as whole cells may be determined by the size of each object located. Like the term “cell objects,” the terms “points of interest” or simply “points” include both whole cells and cell fragments.

A low magnification objective lens (e.g., 4× magnification) may be used to achieve a relatively large field of view and image the whole sample to provide a plurality of low magnification images. Each of the low magnification images may correspond to a different portion of the sample. The low magnification images may be obtained using the non-limiting example embodiment described with reference to FIG. 3.

Each of the low magnification images may be analyzed to determine points of interest in the low magnification images that match a pre-defined cell or cell fragment. Shape(s), color(s), intensity(ies), size limits, etc., may be matched between the low magnification image and the low magnification cell and fragment definition by passing over a matched filter corresponding to the target cell or fragment definition. Matches are detected where the strongest response is found. The filtering also aims to reject debris.

The coordinates of locations with the strongest match may be stored. In one embodiment, a measure of how closely the detected objects match the defined cell or fragment characteristics may also be stored and associated with the respective location.

A matching selection threshold may be adjusted to maintain a desired detection level. The matching selection threshold may be selected by a user input or may be a pre-set value for the optical system, type of sample being images, and/or type of rare cells and fragments being detected. The matching selection threshold may be adjusted in accordance with a user input setting a number of points to collect for each low magnification images or a total number of points to collect in the first stage of the scan. In an example implementation, the number of points to collect may be pre-set based on amount of storage available to store the results of the first stage scan and/or the results of the second stage scan. In an example implementation, the measure of how closely the image at the point of interest matches the definition of the cell or fragment may be used to select which points are retained (e.g., top 50 matching points of interest per image).

At each point of interest in the low magnification image, a thumbnail image may be obtained and stored in storage. The thumbnail image may be a sub-image of the low magnification image. The thumbnail may be associated with other information about the points of interest (e.g., point coordinates, peak intensity, approximate size, and/or intensity of two other wavelengths at same coordinates) that may also be stored. While other example embodiments may store only limited information (e.g., peak intensity, approximate size, and/or intensity of two other wavelengths at same coordinates) about the points of interest, one example embodiment also stores a thumbnail image corresponding to each of the points of interest. The thumbnail images may be associated with the corresponding point of interest. In some example implementations, a single thumbnail image may correspond to a plurality of point of interest if the points of interest are clustered together and the single thumbnail image captures the clustered points of interest.

The process of obtaining low magnification images, selecting points of interest, and obtaining a thumbnail image at each selected point of interest is repeated until the whole sample is scanned and analyzed.

For whole cells, the determined points of interest and the low magnitude thumbnail images may be ranked and/or otherwise selected, and points of interest may be selected from the ranked results. The ranking and/or otherwise selecting may be performed based on the cell definition and/or a defined number of points to retain from the first stage scan. The points of interest may be ranked and/or otherwise select based on a measure of how close the cell definition matches the image at each point of interest. While the points of interest may be ranked and/or otherwise select for one low resolution image, in this step, the points of interest from all of the low magnification images may be ranked and/or otherwise selected to provide a list of points of interest for the whole sample. Other criteria described in this application (e.g., intensity of points of interest and/or diameter of the points of interest) may also be used to rank and/or otherwise select the points of interest. The top points of interest may be selected for further processing based the user input or pre-set values defining a total number of points to select for further processing.

In FIG. 17A, the processing of fragment points of interest from the scan differs from the processing of whole cell points of interest. Because fragments are small they may not all be detected in the low magnification scan images and so a statistical correction factor is applied to those objects found, according to their measured size, e.g., using statistical correction data obtained as in FIG. 15. Although there may be less to be gained from imaging and further matching of characteristics of smaller cell fragments as compared to whole cells, the distribution of sizes of fragments found and the total contribution of fragments in the sample is useful data.

The detected fragments are sorted by size into histogram bins. For each bin, a relevant statistical correction factor is applied to the number in the bin. And for each size, a depth dimension for the fragment is estimated. The depth may be determined for example by calculating an equivalent diameter of a round fragment for each size and then applying this diameter value as an estimate of the depth dimension. An estimated volume is determined using the measured area and the estimated depth of each fragment.

The count of fragments in each histogram bin may then be used as a multiplier to arrive at a total estimated volume of fragments for each size. This total volume for each fragment size may be summed for all fragment sizes to provide an estimate of the total volume of all fragments.

Fragments may potentially bind to antibodies and take up marker fluorophore throughout their volume. An estimate of the total amount (volume) of the fragment is assessed in one example embodiment from a total fluorescent illumination of the fragment. A sum of fragment illuminations from the sample may be an indicator of the total load of cancer derived material.

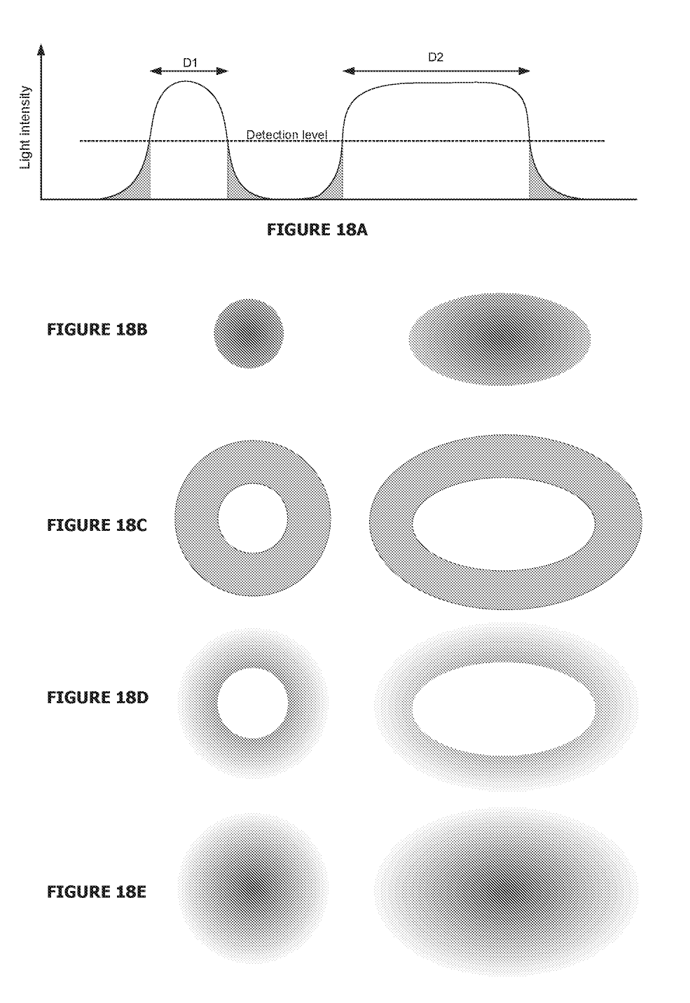

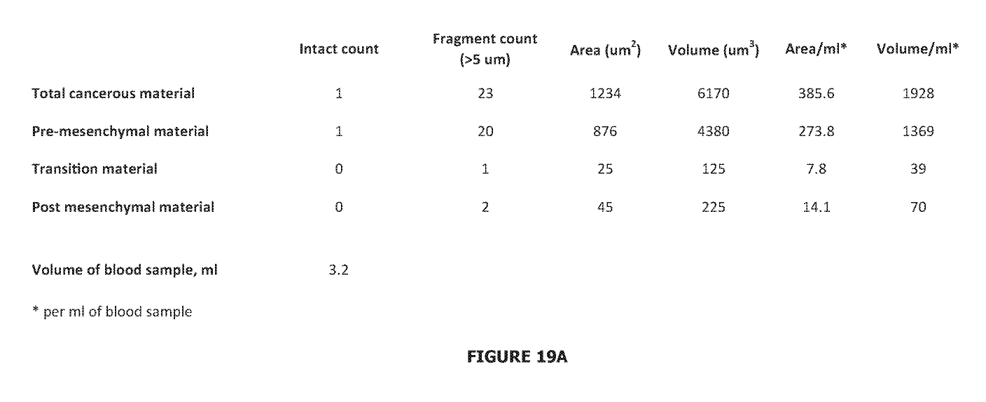

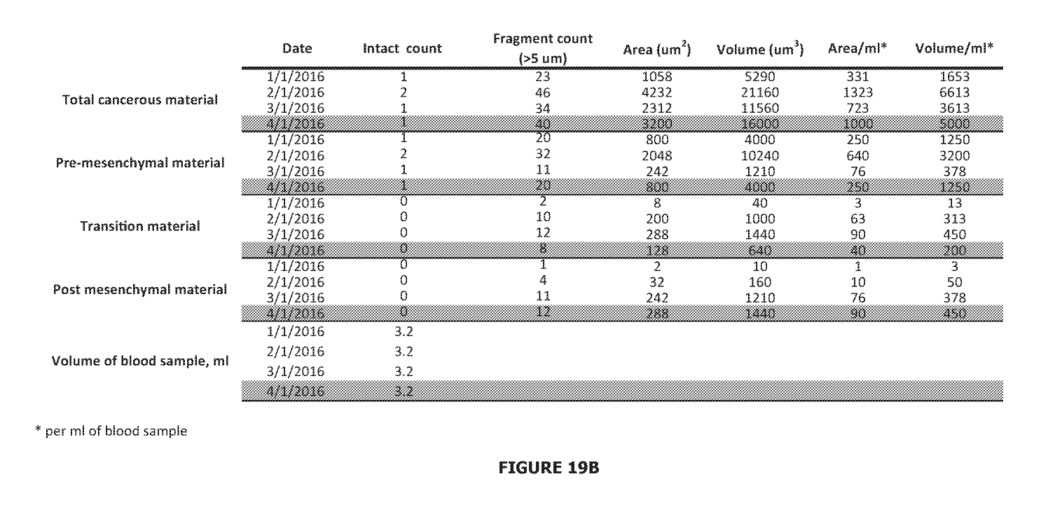

Fragments may be detected by their light intensity in one or more fluorescent channels. In order to measure the diameter of area of fragments an intensity threshold may for example be defined at half the maximum intensity of fragments and the area of the fragment can be estimated from the number of pixels in the fragment image that exceed this threshold. FIG. 18A shows a section through an intensity profile of an image bisecting two small fragments, one larger and one smaller. Because they are small, diffraction in the optics causes the edges to be diffused. However, a half-intensity light intensity detection measurement may be used in one example embodiment to approximate the extent of the fragment. The detected half-intensity level is used to find the periphery of the fragment to measure both the fragment's diameter and area as shown in FIG. 18B.

A total illumination from a fragment may be determined by integrating its intensity over its area, which may be estimated from the sum of the pixel values in the fragment image. However, the pixels included in the measurement area do not need to include all of the light from the fragment. When measuring total illumination from a fragment, pixels in a annulus around the fragment as shown in the example of FIG. 18C are also summed in order to measure light diffracted outside of the nominal fragment area shown in the example of FIG. 18D which forms part of the total fragment illumination shown in the example of FIG. 18E.

Returning to FIG. 17A, after measuring the total illumination or brightness (as referred to in FIG. 17) from each fragment, the individual fragment illuminations or brightnesses are summed for the total fragment illumination from the sample, and an indication of the total cancer material load in the sample is displayed and stored.

The histogram of fragment sizes (e.g., a histogram with size along the horizontal axis and frequency of occurrence on the vertical axis) and the total estimated volume of all fragments can also be presented to the user and saved in memory. Other statistical displays may be used with some other examples being described later.

The parallel processing of full cells in FIG. 17B follows a different route from after scanning. Whole cell images are more likely to convey useful information about the cell than cell fragments. As a result, after ranking and selecting of points, the computer controller selects a number of points of interest for detailed imaging. These detailed images may be ranked using the same cell definition, but with more precision because these are higher resolution images. The ranking of these more detailed images may be presented in a histogram with score or rank along the horizontal axis ranging from totally unlike to identical to the cell definition and with count of cells of each score or rank on the vertical axis. Another example statistical analysis summarizes numerically how many cells appear to be pre- or post-mesenchymal transition as indicated by a detected ratio of cytokeratin to vimentin.

Such statistic calculations resulting from the processes in FIGS. 17A and 17B may be used to reduce the number of high resolution images that are required to be taken and saved. Some of these statistical data come from the low magnification scan (e.g., as described above for finding and summing fragments using low magnification) while some statistical data come from the high resolution images, e.g., the scoring and identification of whole cells. Thus, the display and store cell images and statistics block is where data is collected from the low and high magnification scans, summarized into useful human-readable form, and presented to a user on the computer display. When storing the results of the process, e.g., high resolution images, other data including these statistics may also be stored.

The results of the ranking and selected points in FIGS. 17A and 17B may be displayed on a display coupled to the one or more processors. The results may for example be displayed in a table with each entry of a point of interest being provided with information about the point of interest (e.g., coordinates or intensity level) and/or the thumbnail image of the point of interest. The thumbnail images may be displayed in a linear sequence (e.g., as a filmstrip) so that they can be quickly visually inspected. The sequence of the thumbnails may be updated if the criteria used to sort the points of interest is changed and/or points of interest are removed from the table. The user may be provided with a user interface to select which information is displayed with each entry in the table. At this point, a user may also perform a visual inspection of the captured thumbnail images to identify points of interest that should be discarded (e.g., points of interest with debris).

A high magnification image may be obtained at each of the remaining points of interest. The stage may be moved such that the high magnification image may be obtained at the coordinates of the point of interest. The high magnification image may be obtained with higher magnification objective as compared to the low magnification image. For example, the higher magnification objective may be a 40× objective lens.

The obtained high magnification images may be displayed on the display and one or more user inputs may select the images to be retained and/or removed. In one embodiment, the high magnification images may be displayed along with the corresponding low resolution thumbnail images and/or other information about the corresponding point of interest so that the user may make a more informed selection of the images with the rare cells. In another embodiment, user inputs may select which high magnification images should be discarded and/or retained. In one embodiment, a user may define criteria and/or number of image to retain and the high magnification images matching the defined criteria may be retained and/or the defined number of top ranked images may be retained.

The selected images along with the related statistical data for a sample may be stored in memory coupled to the one or more processors and/or transmitted (e.g., over a network) to other storage. The selected high magnification images may be stored in association with the points of interest and/or the thumbnail images.

FIG. 19A shows an example output from one rare cell scan displayed in table form and illustrating cell count, fragment count, as well as area in two different units of measure, volume in two different units of measure for total rare cell or disease-indicative material, pre-mesenchymal material, transition material, post-mesenchymal material, and volume of the sample.

FIG. 19B shows another example where chronological results of individual scans are collated into one table illustrating cell count, fragment count as well as area in two different units of measure and volume in two different units of measure for total rare cell disease indicative material, pre-mesenchymal material, transition material, post-mesenchymal material, and volume of the sample. This format shows more clearly how the measurements have evolved over time for multiple dates.

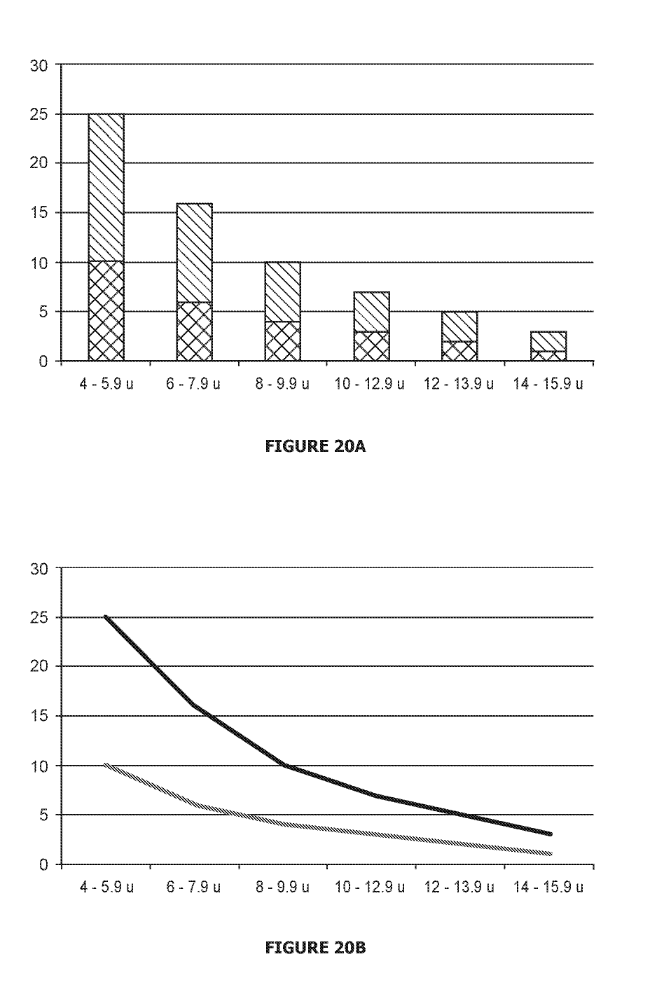

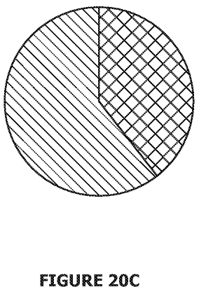

FIGS. 20A and 20B show further example output displays for illustrating ratios and sizes of detected cell load per sample for epithelial cell load and metastatic cell load in bar graph, and curve display formats. Other display formats may be used such as 20C where a simple ratio of metastatic to epithelial cells is displayed. In FIGS. 20A and 20C, the diagonal lines represent epithelial cell load and the cross-hatching represents metastatic cell load. In FIG. 20B, the dark line graphs epithelial cell load against cell fragment size, and the light line graphs metastatic cell load against cell fragment size.

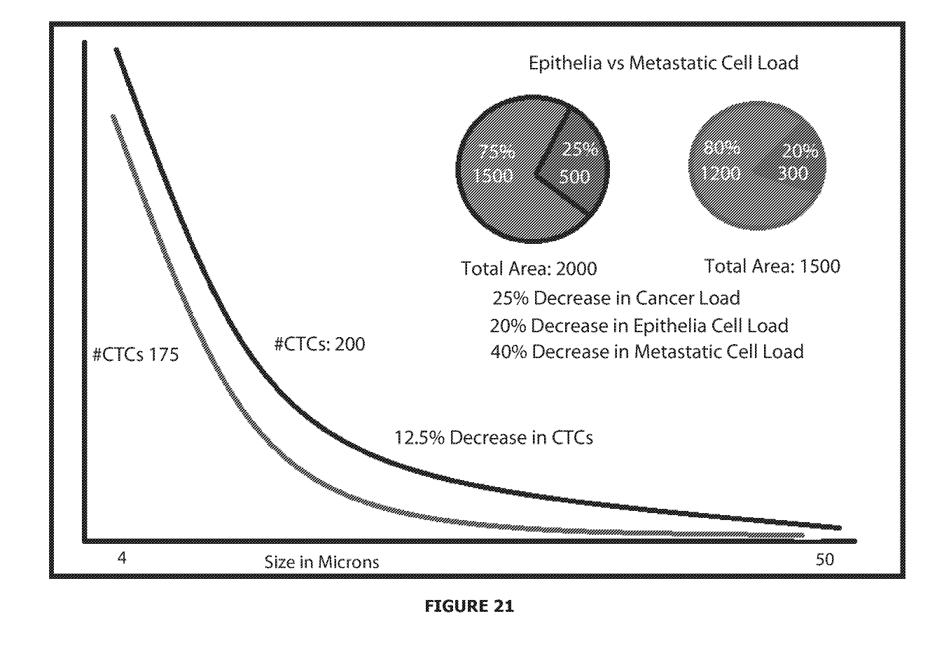

One example of another format is shown in FIG. 21 which summarizes and compares epithelia vs. metastatic cell load for two samples taken at different points in time using pie charts, numbers, and text. FIG. 21 includes a graph that plots cell load against cell fragment size for each measurement. The dark line graph and left side pie chart are from an earlier date and are shown compared against the lighter line graph and right hand pie chart from a later or latest measurement.

In the embodiments discussed above, the cell fragments and/or whole cells results of the first stage scan and/or the second stage scan are sorted to provide points of interest that are more likely to be target rare cells. An expert may observe the top ranked results to make a decision or diagnosis. Accurately-ranked results may allow an expert who interprets the data to review only the highest ranked images in order to reach a conclusion about the sample, although other images are also retained and available for the expert to observe if desired.

Although the present disclosure has been described with reference to particular example embodiments, it will be appreciated by those skilled in the art that the disclosure may be embodied in many other forms.

All methods described herein can be performed in any suitable order unless otherwise indicated herein. The use of any and all examples, or example language (e.g., “such as”) provided herein, is intended merely to better illuminate the example embodiments and does not pose a limitation on the scope of the claims appended hereto unless otherwise claimed. No language or terminology in this specification should be construed as indicating any non-claimed element as essential or critical.

Throughout this specification, unless the context requires otherwise, the word “comprise”, or variations such as “comprises” or “comprising” “including,” “containing,” and the like will be understood to imply the inclusion of a stated element or integer or group of elements or integers but not the exclusion of any other element or integer or group of elements or integers.

As used herein, the singular forms “a,” “an,” and “the” may also refer to plural articles, i.e., “one or more,” “at least one,” etc., unless specifically stated otherwise. For example, the term “a fluorophore” includes one or more fluorophores.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller subranges are also included. The upper and lower limits of these smaller ranges are also included therein, subject to any specifically excluded limit in the stated range.

The term “about” or “approximately” means an acceptable error for a particular recited value, which depends in part on how the value is measured or determined. In certain embodiments, “about” can mean 1 or more standard deviations. When the antecedent term “about” is applied to a recited range or value it denotes an approximation within the deviation in the range or value known or expected in the art from the measurements method. For removal of doubt, it shall be understood that any range stated herein that does not specifically recite the term “about” before the range or before any value within the stated range inherently includes such term to encompass the approximation within the deviation noted above.

1. Apparatus for detecting the presence of marked cell objects contained in a sample, comprising:
an optical system configured to optically scan the sample in a first optical operation during a first time period to generate a first set of image data, and
data processing circuitry configured to detect, from the first set of image data, marked cell objects in the sample, determine one or more parameters associated with a detected marked cell object, and generate coordinate locations of detected marked cell objects in the sample,
wherein the detected marked cell objects include at least a plurality of cell fragments, each of the cell fragments being smaller than a whole cell.

2. An apparatus as in claim 1, wherein the one or more parameters includes shape, color, intensity, or size.

3. An apparatus as in claim 1, wherein the data processing circuitry is configured to determine cell fragment count information for the sample and to generate output information based on the cell fragment count information.

4. An apparatus as in claim 1, wherein the data processing circuitry is configured to aggregate information for detected cell fragments for the sample and to output the aggregate information as an indicator of disease progression.

5. An apparatus as in claim 4, wherein the data processing circuitry is configured to generate circulating tumor or cancer cell load information from the aggregate information.

6. An apparatus as in claim 1, wherein the data processing circuitry is configured to determine an area associated with each of the plurality of cell fragments, combine the determined cell fragment areas, and divide the combination by a representative whole cell area to generate equivalent cell load information for output.

7. An apparatus in claim 6, wherein the data processing circuitry is configured to determine the area of each of the cell fragments using a fractional-intensity detection measurement of the cell fragment.

8. An apparatus in claim 1, wherein the data processing circuitry is configured to determine a volume associated with each of the plurality of cell fragments, combine the determined cell fragment volumes, and divide the combined cell fragment volumes by a representative whole cell volume to generate equivalent cell load information for output.

9. An apparatus in claim 8, wherein the data processing circuitry is configured to estimate a cell fragment volume depth.

10. An apparatus in claim 8, wherein the data processing circuitry is configured to determine the volume of each of the cell fragments using a light intensity detection measurement of the cell fragment.

11. An apparatus as in claim 1, further comprising:
a memory, coupled to the data processing circuitry, configured to store determined cell object parameter information and coordinate location information associated with the coordinate locations of at least some of the detected marked cell objects,
wherein the optical system is configured, in a second optical operation during a second time period, to obtain image data at the coordinate locations of selected ones of the detected marked cell objects,
wherein the data processing circuitry is configured to process the obtained image data to characterize at least some of the selected marked cell objects and generate output information based on the characterization of the selected marked cell objects.

12. An apparatus as in claim 11, wherein the data processing circuitry is configured to determine cell fragment count information for the sample during the first optical operation and to selectively perform the second optical operation based on the determined cell fragment count information for the sample.

13. An apparatus as in claim 1, wherein the data processing circuitry is configured to determine cell fragment count information and whole cell count information for the sample and to generate output information based on the determined cell fragment count information and the determined whole cell count information.

14. An apparatus as in claim 1, wherein the data processing circuitry is configured to generate thumbnail image files for detected cell fragments for the sample during the first optical operation.

15. An apparatus as in claim 1, wherein the data processing circuitry is configured to analyze detected cell fragments for the sample to determine a degree of match between detected cell fragments for the sample and a predetermined cell fragment definition.

16. An apparatus as in claim 15, wherein the data processing circuitry is configured to perform a filtering operation when determining the degree of match.

17. An apparatus as in claim 15, wherein the data processing circuitry is configured to rank or select certain ones of the detected cell fragments based on how close the detected cell fragments match the predetermined cell fragment definition.

18. An apparatus as in claim 3, wherein the data processing circuitry is configured to:
calibrate the apparatus using a distribution of different uniform size microspheres and to generate statistically-based correction factors for different fragment sizes using scans of the different uniform size microspheres by the optical system, and
compensate the determined cell fragment count information for the sample using the statistically-based correction factors for different fragment sizes.

19. Apparatus for detecting the presence of marked cell objects contained in a sample, comprising:
an optical system configured to optically scan the sample in a first optical operation during a first time period to generate a first set of image data, and
data processing circuitry configured to:
detect, from the first set of image data, marked cell objects in the sample,
determine one or more parameters associated with a detected marked cell object,
determine aggregate information for detected marked cell objects for the sample, and
output the determined aggregate information,
wherein the determined aggregate information is indicative of progression of disease.

20. An apparatus in claim 19, wherein the disease is cancer.

21. An apparatus in claim 19, wherein the determined aggregate information includes one or more ratios associated with epithelial cell load and metastatic cell load for detected marked cell objects

22. An apparatus as in claim 19, wherein the determined aggregate information includes an aggregate area associated with the detected marked cell objects for the sample.

23. An apparatus as in claim 19, wherein the determined aggregate information includes an aggregate volume associated with the detected marked cell objects for the sample.

24. An apparatus as in claim 19, wherein the determined aggregate information includes an aggregate brightness value associated with the detected marked cell objects for the sample.

25. An apparatus as in claim 19, wherein the data processing circuitry is configured to determine and generate the output information for the sample for different times.

26. An apparatus as in claim 19, wherein the detected marked cell objects include at least a plurality of cell fragments, each of the cell fragments being smaller than a whole cell, and wherein the data processing circuitry is configured to generate and display a histogram of different size cell fragments detected in the sample.

27. An apparatus as in claim 26, wherein the data processing circuitry is configured to generate and output total cell fragment volume information associated with the sample.

28. A method for detecting the presence of marked cells in a sample of cells contained, comprising:
a) in a first optical operation, optically scanning the sample during a first time period to generate a first set of image data,
b) from the first set of image data, detecting marked cell objects in the sample of cells,
c) determining one or more parameters associated with detected marked cell objects, and
d) generating coordinate locations of detected marked cell objects in the sample,
wherein the detected marked cell objects include at least a plurality of cell fragments, each of the cell fragments being smaller than a whole cell,

29. The method as in claim 28, further comprising:
determining cell fragment count information for the sample, and
generating output information based on the cell fragment count information.

30. The method in claim 29, further comprising:
aggregating information for detected cell fragments for the sample, and
outputting the aggregate information as an indicator of disease progression.

31. The method as in claim 28, further comprising:
determining an area associated with each of the plurality of cell fragments,
combining the determined cell fragment areas, and
dividing the combination by a representative whole cell area to generate equivalent cell load information for output.

32. The method as in claim 28, further comprising:
determining a volume associated with each of the plurality of cell fragments,
combining the determined cell fragment volumes, and
dividing the combined cell fragment volumes by a representative whole cell volume to generate equivalent cell load information for output.

33. The method as in claim 28, further comprising:
saving in memory determined cell object parameter information and coordinate location information associated with the coordinate locations of at least some of the detected marked cell objects,
in a second optical operation during a second time period, obtaining image data at each of the coordinate locations of selected ones of the detected marked cell objects,
processing the obtained image data to characterize at least some of the detected marked cells,
generating output information based on the characterization of the selected marked cell objects, and
determining cell fragment count information for the sample during the first optical operation and to selectively perform the second optical operation based on the determined cell fragment count information for the sample.

34. A method for detecting the presence of marked cell objects contained in a sample, comprising:
a) in a first optical operation, optically scanning the sample during a first time period to generate a first set of image data,
b) from the first set of image data, detecting marked cell objects in the sample of cells,
c) determining one or more parameters associated with detected marked cell objects, and
d) determining aggregate information for detected marked cell objects for the sample, and
e) output the determined aggregate information,
wherein the determined aggregate information is indicative of progression of disease.

35. A method in claim 34, wherein the disease is cancer.

36. A method in claim 34, wherein the determined aggregate information includes one or more ratios associated with epithelial cell load and metastatic cell load for detected marked cell objects

37. A method in claim 34, wherein the determined aggregate information includes an aggregate area associated with the detected marked cell objects for the sample.

38. A method in claim 34, wherein the determined aggregate information includes an aggregate volume associated with the detected marked cell objects for the sample.

39. A method in claim 34, wherein the determined aggregate information includes an aggregate brightness value associated with the detected marked cell objects for the sample.

40. The method in claim 34, further comprising determining and generating the output information for the sample for different times.

41. The method in claim 34, wherein the detected marked cell objects include at least a plurality of cell fragments, each of the cell fragments being smaller than a whole cell, and the method further comprises generating and displaying a histogram of different size cell fragments detected in the sample.
